(19) United States
(12) Patent Application Publication
(10) Pub. No.: US 2026/0185052 A1
(43) Pub. Date: Jul. 2, 2026

(54) SURFACE MODIFIED HYDROGELS COMPRISING VIABLE CELLS, METHODS OF MANUFACTURE AND USE THEREOF

(71) Applicant: THE STATE OF ISRAEL, MINISTRY OF AGRICULTURE & RURAL DEVELOPMENT, AGRICULTURAL, Rishon Lezion (IL)

(72) Inventors: Hinanit KOLTAI, Rishon LeTsiyon (IL); Guy MECHREZ, Adi (IL); Nurit SHALEV, Tirat Yehuda (IL); Avital BENHAIM, Rosh Ha'ayin (IL)

(21) Appl. No.: 19/109,219

(22) PCT Filed: Sep. 6, 2023
(86) PCT No.: PCT/IL2023/050962

§ 371 Date: Mar. 6, 2025

Related U.S. Application Data
(60) Provisional application No. 63/403,982, filed on Sep. 6, 2022.

(51) Int. Cl. C12N 5/0775

(52) U.S. Cl. CPC C12N 5/0668; C12N 2501/105; C12N 2501/11; C12N 2501/115; C12N 2501/15; C12N 2531/00; C12N 2533/54; C12N 2533/56; C12N 2533/74; C12N 2533/80; C12N 2537/10

(57) ABSTRACT

Provided herein is a composition comprising a hydrogel covalently bound to a functional moiety, wherein the hydrogel comprises a cross linkable biopolymer and a first cell population; the cross linkable biopolymer is at least partially crosslinked by a crosslinking agent within the hydrogel; and the functional moiety has a binding affinity to a cell. Furthermore, a method of growing cells using the composition of the invention, and a method of manufacturing the composition using a kit disclosed herein are also provided.

### CROSS REFERENCE TO RELATED APPLICATIONS

This application claims the benefit of priority to U.S. Provisional Patent Application No. 63/403,982 filed 6 Sep. 2022, entitled “SURFACE MODIFIED HYDROGELS COMPRISING VIABLE CELLS, METHODS OF MANUFACTURE AND USE THEREOF” the contents of which are incorporated herein by reference in their entirety.

### FIELD OF THE INVENTION

The present invention is directed to the surface modified hydrogel-based compositions comprising viable cells, methods of manufacturing and using the same such as for supplementing cells with an active agent.

### BACKGROUND OF THE INVENTION

A 2020 analysis indicated that 55-95% of the cost of cultured meat production could be attributed to the cost of growth media. Moreover, of that cost, 99% is due to the price of growth factors with Transforming growth factor (TGF)-β accounting for the majority of that cost. While it is difficult to find a consistent cost estimate for a lab-grown burger today, there are estimates of between $10-50 per pound or €9 for a burger.

Accordingly, in order to obtain a cost efficient cultured meat production, there is a need for a new technology for a specific delivery systems capable of significantly reducing the amount of growth factors utilized for the manufacturing of cultured meat.

### SUMMARY OF THE INVENTION

In one aspect of the invention, there is provided a composition comprising a scaffold covalently bound to a functional moiety, wherein the scaffold comprises a cross linkable biopolymer and a first cell population; the cross linkable biopolymer is at least partially crosslinked by a crosslinking agent within the scaffold; the functional moiety has a binding affinity to a second cell population; and wherein the functional moiety comprises a hyaluronic acid including any salt and any copolymer thereof.

In one embodiment, the cross linkable biopolymer comprises (i) one or more of a cross-linkable polysaccharide, (ii) one or more of a cross-linkable protein or both (i) and (ii), wherein any of (i) and (ii) includes any salt, any conjugate, or any hydrolysate thereof.

In one embodiment, the cross-linkable polysaccharide is selected from alginic acid, chitosan, gellan gum, dextran, agarose, and carrageenan; and wherein the cross-linkable protein is selected from gelatin, collagen, fibrinogen, vitronectin, laminin and fibronectin including any salt, any conjugate, or any hydrolysate thereof.

In one embodiment, the second cell population is attached to the scaffold via the hyaluronic acid.

In one embodiment, covalently bound is via an amide bond, via an ester bond, a linker, a click reaction product, a thioether bond, a linker, or any combination thereof.

In one embodiment, a w/w ratio between the cross linkable polysaccharide and the cross linkable protein within the scaffold is between about 1:1 and about 1:3.

In one embodiment, the crosslinking agent is selected from thrombin, a multivalent metal cation, a polyanion, and a chemical cross-linker, including any salt and any combination thereof.

In one embodiment, the cross-linkable biopolymer comprises alginic acid, gelatin, collagen and fibrinogen including any salt thereof; and wherein the crosslinking agent comprises thrombin and the multivalent metal cation; wherein the multivalent metal cation is or comprises Ca2+.

In one embodiment, the scaffold is a hydrogel.

In one embodiment, the scaffold is characterized by a predefined pattern on a support or by a predefined shape, and wherein the functional moiety is bound to at least a portion of the outer surface of the predefined pattern or of the predefined shape.

In one embodiment, the predefined shape comprises a particle characterized by an average particle size between 100 μm and 1 cm. In one embodiment, the particle is a substantially spherical particle

In one embodiment, the first cell population is embedded into or is located inside the particle; wherein the first cell population comprise mammalian cells being in a form of a cell culture, a tissue, or both; and wherein the mammalian cells are configured to secret an active agent.

In one embodiment, the active agent comprises a small molecule, a growth factor, a differentiation factor, a polyamino acid, a polynucleic acid, or any combination, thereof; and wherein the active agent is essential for activity of the second cell population.

In one embodiment, the growth factor comprises any one of Transforming growth factor beta (TGF-β); Insulin-like growth factor 1; fibroblast growth factor2; epidermal growth factor; interleukin 6; Neuregulin 1, or any combination thereof.

In one embodiment, the growth factor is TGF-β and wherein the mammalian cells comprise TGF-β secreting cells.

In one embodiment, the TGF-β secreting cells comprise any one of a macrophage, an epithelial cell, and a monocyte, including any combination thereof; and wherein the macrophage comprises a differentiated macrophage or a non-differentiated macrophage.

In one embodiment, the second cell population comprises mammalian cells selected from any one of: Embryonic stem cells, Induced pluripotent stem cells, mesenchymal stem cells, Fibro-adipogenic progenitor cells, Muscle satellite cells, Fibroblasts, Endothelial cell, and Smooth muscle cells, including any combination thereof.

In another aspect, there is provided a method for delivering an active agent to a cell, comprising contacting the composition of the invention with the cell, under conditions suitable for binding of the cell to an outer portion of the composition; and wherein the plurality of viable cells of the composition are configured to secret said active agent; and wherein the active agent is essential for activity of the cell.

In one embodiment, the plurality of cells comprise mammalian cells; and wherein the active agent comprises a small molecule, a growth factor, a polyamino acid, a polynucleic acid, or any combination, thereof.

In one embodiment, the growth factor comprises any one of Transforming growth factor beta (TGF-β); Insulin-like growth factor 1; fibroblast growth factor2; epidermal growth factor; interleukin 6; Neuregulin 1, or any combination thereof.

In one embodiment, the growth factor is TGF-β and wherein the cell is selected from any one of: Embryonic stem cells, Induced pluripotent stem cells, mesenchymal stem cells, Fibro-adipogenic progenitor cells, Muscle satellite cells, Fibroblasts, Endothelial cell, and Smooth muscle cells, including any combination thereof.

In another aspect, there is provided a kit comprising (i) alginic acid, including any salt, or any conjugate thereof; (ii) one or more of a cross linkable protein, and (iii) a crosslinking agent; wherein the kit comprises a first compartment comprising the (i) and (ii); a second compartment comprising the (iii); the (i), the (ii) and the (iii) are each in a form of a flowable aqueous composition; the kit further comprises a functional moiety having a binding affinity to a second cell population; and the kit optionally comprises a first cell population configured to secret an active agent.

In one embodiment, a w/w ratio between (i) and (ii) within the kit is between about 1:1 and about 1:3.

In one embodiment, wherein the (i) and the (ii) are mixed together with the first cell population within the first compartment.

In one embodiment, the kit further comprises instructions for contacting the first compartment and the second compartment at a predetermined ratio, to obtain a crosslinked hydrogel; and wherein the first cell population is embedded within the crosslinked hydrogel.

In one embodiment, the functional moiety comprises hyaluronic acid; and wherein the functional moiety is stored within a third compartment further comprising a sufficient amount of an activating agent and wherein the contacting is performed by printing.

In one embodiment, the activating agent is configured to promote a covalent bonding between the functional moiety and the crosslinked hydrogel; and wherein the kit further comprises instructions for contacting the third compartment with the crosslinked hydrogel, to obtain the functional moiety covalently bound to the crosslinked hydrogel; wherein an amount of the functional moiety is between 1 ng and 1 mg per 10 mg of the crosslinked hydrogel.

In one embodiment, the functional moiety comprises a carboxy group; and wherein the activating agent is configured to modify the carboxy group into an active ester.

In one embodiment, the crosslinking agent is selected from thrombin, fibrinogen, a multivalent metal cation, a polyanion, and a chemical cross-linker, including any salt and any combination thereof.

In one embodiment, the multivalent metal cation comprises Ca2+.

In one embodiment, the one or more of a cross linkable protein comprises collagen, gelatin and fibrinogen, and optionally further comprises at least one protein selected from vitronectin, laminin and fibronectin, including any salt, any conjugate, or any combination thereof; and wherein the crosslinking agent comprises Ca2+ and thrombin.

In one embodiment, a w/w ratio between collagen and fibrinogen within the kit is between about 1:10 and 1:50.

In one embodiment, the first compartment comprises (i) alginic acid and gelatin, each independently at a concentration between about 1 and 10% w/w; and (ii) collagen and fibrinogen, wherein a concentration of fibrinogen is between about 0.1 and about 5% w/w; and the first cell population.

In one embodiment, the second compartment comprises Ca2+ at a concentration between 10 and 200 mM and thrombin at a concentration between 500 and 5000 U/ml.

In another aspect, there is provided a crosslinked hydrogel obtained by contacting the first compartment and the second compartment of the kit of the invention, wherein the crosslinked hydrogel comprises a first cell population configured to secret an active agent; and wherein the first cell population is embedded within the crosslinked hydrogel.

In one embodiment, the crosslinked hydrogel in a form of a particle or in a form of a pattern on top of a substrate.

In one embodiment, the particle is characterized by a predefined shape and further comprising a functional moiety covalently bound to the particle; and wherein the functional moiety has a binding affinity to a second cell population.

In one embodiment, the functional moiety comprises hyaluronic acid, including any salt thereof; and wherein the second cell population is the second cell population of the invention.

In one embodiment, the particle is obtained by contacting the particle of the invention with an activated hyaluronic acid.

In one embodiment, the contacting is performed via a printing method.

In one embodiment, the crosslinked hydrogel is the scaffold of the invention.

In one embodiment, an amount of the first cell population within the particle is between 1000 and 200000 cell units.

In one embodiment, an amount of the second cell population within the particle is between 500 and 2000000 cell units.

In one embodiment, the functional moiety is hyaluronic acid, and wherein an amount of hyaluronic acid relative to the hydrogel is between 0.1 ng and 0.1 mg per 1 mg of the hydrogel.

In another aspect, there is provided a crosslinked hydrogel obtained by contacting the first compartment and the second compartment of the kit of the invention, wherein the crosslinked hydrogel comprises a plurality of viable cells.

In another aspect, there is provided a system comprising a kit of the invention and a printing apparatus. In one embodiment, the printing apparatus is a bioprinting device.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 present a schematic illustration of an exemplary scaffold of the invention comprising (A) bovine cells (first cell population) producing and secreting growth factor(s) and differentiation factor(s); (B) hyaluronic acid (HA) covalently bound to a surface of the scaffold and promotes attachment of the second cell population (C), such as Bovine mesenchymal stem cells (BMSC) which form a basis for cultured meat.

FIG. 2 is a micrograph showing BMSC cell culture. Black arrow point to mesenchymal cells, red arrow to myotubes that differentiate from the mesenchymal cells.

FIG. 3A-3B are micrographs showing printed microparticles of the invention (AGFTC Beads) decorated with 6-aminofluorescein labeled HA bound to their surface. (3A) AGFTC Beads with CRL-1390 cells at the day of printing. (3B) shows CRL-1390 cells proliferating within the AGFTC beads.

FIG. 4 presents micrographs showing AGFTC beads surface modified with 6-amino fluorescein labeled hyaluronic acid in bright field (left image) and in green fluorescence channel (right image). AGFTC beads were covalently bonded to 6-AF-labeled HA using the EDC reaction to confirm successful covalent attachment. Bar corresponds to 100 μm.

FIG. 5 is a bar graph showing enhanced production of growth factors (TGF1β) by CRL-1390 and CRL-2048 in culture.

FIG. 6 is a micrograph showing examples for BMSC cells attached to AGFTC bead that start the process of differentiation (yellow arrows). Blue arrow denote the AGFTC bead.

### DETAILED DESCRIPTION OF THE INVENTION

According to some embodiments, the present invention provides a composition comprising a crosslinked cell scaffold comprising a plurality of viable cells, and wherein the crosslinked cell scaffold is covalently bound to a functional moiety having a binding affinity to a cell. In some embodiments, the crosslinked cell scaffold is a biopolymer-based matrix comprising the plurality of viable cells, as disclosed herein. In some embodiments, the crosslinked cell scaffold comprises one or more cross linkable biopolymer, wherein the one or more cross linkable biopolymer is/are at least partially crosslinked within the crosslinked cell scaffold. In some embodiments, the crosslinked cell scaffold is characterized by a predefined shape or is in a form of a pattern. In some embodiments, the functional moiety is or comprises a ligand capable of binding to a cellular receptor (e.g. a cell surface receptor). In some embodiments, the functional moiety is covalently bound to the outer surface of the crosslinked cell scaffold. The outer surface refers to the portion of the scaffold facing an ambient (e.g. a cell medium), and the inner portion refers to the portion of the scaffold facing the viable cells. In some embodiments, the cross linkable biopolymer comprises a cross-linkable polysaccharide, a cross-linkable protein, or both, including any salt, any conjugate, any co-polymer, or any hydrolysate thereof.

The terms “crosslinked cell scaffold” and “scaffold” are used herein interchangeably. The term “binding” encompasses a stable cell attachment to the functional moiety (and as a consequence to the outer surface of the scaffold), wherein “stable” refers to a sufficient binding affinity between functional moiety having a binding affinity to a cellular receptor so that the cell remains attached to the functional moiety and/or to the scaffold under conditions suitable for growing the cell (such as aqueous cell culture medium).

In some embodiments, the plurality of viable cells are embedded within the crosslinked cell scaffold. In some embodiments, the plurality of viable cells are bound to the crosslinked cell scaffold. In some embodiments, the plurality of viable cells are encapsulated within the crosslinked cell scaffold. In some embodiments, the plurality of viable cells are homogenously distributed within the crosslinked cell scaffold. In some embodiments, the plurality of viable cells are agglomerated (form colonies) within the scaffold. In some embodiments, the composition of the invention is a composite, comprising the plurality of viable cells homogenously distributed within the crosslinked cell scaffold.

In some embodiments, the crosslinked cell scaffold is a matrix. In some embodiments, the matrix is a three-dimensional matrix comprising at least partially cross-linked biopolymer(s). In some embodiments, the crosslinked cell scaffold is a bulk material (e.g. a composite material). In some embodiments, the crosslinked cell scaffold is a solid at a predefined temperature of up to 60 C, up to 50 C, or up to 40 C, including any range between.

The term “solid” refers to the ability of a material (such as scaffold or hydrogel disclosed herein) to maintain its 3D shape or pattern at a predefined temperature under predefined conditions including ambient atmosphere, and/or humidity, and/or aqueous environment. A skilled artisan will appreciate that a solid may undergo swelling upon contacting thereof with water, thus the term “solid” encompasses that a swollen material maintains its shape in an aqueous environment. Alternatively, the term “solid” encompasses that a dry material (i.e. non-swollen substantially dry material) maintains its shape in a non-humid environment (or in a non-aqueous environment), such as an ambient atmosphere having a humidity below 80%, below 70%, or below 50%. As used herein, the term “maintain” including any grammatical form thereof, refers to at least partial shape retention, so that the material doesn't undergoes disintegration and is still defined by a 3D geometrical shape or by a specific pattern.

In some embodiments, the crosslinked cell scaffold is a dry solid. In some embodiments, a water content of the dry solid is below 20%, below 15%, below 10%, below 5%, below 1%, or between 1 and 10%, between 1 and 15%, or between 5 and 20% by weight of the crosslinked cell scaffold, including any range between. In some embodiments, the dry solid is swellable, configured to uptake at least 50%, at least 100%, at least 500%, at least 1000%, at least 5000% water relative to its initial weight (i.e. the weight of the dry solid).

In some embodiments, the composition and/or the crosslinked cell scaffold is/are grade product(s). As used herein, the term “food grade” refers to a product consisting of food-grade ingredients approved for human consumption by a corresponding regulatory authority (i.e., GRAS). The concentration of each of the constituents within the food grade doesn't exceed a toxicity limit for the specific constituent as determined by the corresponding regulatory authority.

### Hydrogel

In some embodiments, the crosslinked cell scaffold is a gel. In some embodiments, the crosslinked cell scaffold is a hydrogel. In some embodiments, the hydrogel comprises one or more cross linkable biopolymer (i.e. matrix) and water, wherein the biopolymer is as described herein. In some embodiments, the hydrogel is a solid.

In some embodiments, the crosslinked cell scaffold in a form of a hydrogel comprising (or consisting essentially of) a cross linkable biopolymer and further comprises between 10 and 95% water, between 10 and 50% water, between 10 and 30% water, between 10 and 40% water, between 10 and 60% water by weight of the crosslinked cell scaffold, including any range between.

In some embodiments, the crosslinked cell scaffold (e.g. in a form of a hydrogel) comprises a matrix of self-assembled cross linkable biopolymer(s) encapsulating the plurality of viable cells. In some embodiments, the cross linkable biopolymer(s) is at least partially cross-linked within the crosslinked cell scaffold. In some embodiments, crosslinked is via a physical cross-link.

The term “cross-linking” as used herein refers to the formation of a bond between two molecules, wherein the bond refers to a physical bond (a non-covalent bond, such as an electrostatic interaction, a Van-der-Waals interaction, a dipol-dipol interaction, hydrogen bond, pi-pi stacking, etc.), or to a chemical bond. In some embodiments, cross-linking comprises inter cross-linking. In some embodiments, cross-linking comprises intra cross-linking. In some embodiments, cross-link is formed via a cross-linking agent.

As used herein, the term “matrix” refers to a network of biopolymer chains that are randomly distributed therewithin. Matrix further comprises pores (void spaces filled with a gas). Matrix may include a single layer or a plurality of chemically/structurally distinct layers, and may further include any material incorporated within the matrix and/or interposed between the layers or between. In some embodiments, each biopolymer chain within the matrix is in contact with at least one additional biopolymer chain. In some embodiments, the biopolymer chains are randomly distributed within the matrix, to obtain a three-dimensional mesh structure comprising a void space between the chains. In some embodiments, the biopolymer chains are randomly distributed within the matrix thus forming an intertwined polymeric mesh further comprising a plurality of pores. In some embodiments, the matrix is substantially devoid of polymeric chains aligned or oriented in a specific direction.

The term “layer” refers to a substantially homogeneous substance of substantially uniform-thickness which maintains its physico-chemical properties (e.g., glass transition temperature, Youngs modulus, elongation) with the entire dimensions (lengths and width dimensions) thereof. In some embodiments, each layer has a different physical structure and/or a different chemical composition. In some embodiments, each layer has the same physical structure and/or the same chemical composition. In some embodiments, the term “layer”, refers to a polymeric layer.

According to another aspect of the invention, there is provided a composition comprising the scaffold of the invention in a form of a hydrogel covalently bound to a functional moiety, wherein the hydrogel comprises a cross linkable biopolymer and a plurality of viable cells; the cross linkable biopolymer is at least partially crosslinked by a crosslinking agent within the hydrogel; and the functional moiety has a binding affinity to a cell(s). In some embodiments, the terms “scaffold” and “hydrogel” are used herein interchangeably. In some embodiments, the scaffold in a form of a hydrogel is a solid. In some embodiments, the scaffold is compatible with a cell culture. In some embodiments, the term “compatible” encompasses that the scaffold promotes or enables cell attachment thereto and further enables cell growth or cell proliferation on the scaffold. In some embodiments, the “cell growth or cell proliferation” refers to the ability of cells to proliferate or to remain viable under conditions suitable for cell culture (such as cell culture medium, a temperature of about 37° C., and an atmosphere suitable for cell culture), wherein the term “viable” encompasses the definition of a “live cell” below. In some embodiments, the scaffold is compatible with a cell culture of the second cell population, disclosed below (e.g. any mammalian cell type used for the manufacturing of cultured meat).

In some embodiments, the term “hydrogel” refers to a non-Newtonian fluid comprising a supramolecular structures of self-assembled biopolymer molecules (e.g., the cross-linkable protein and/or cross-linkable polysaccharide) and water. In some embodiments, the hydrogel of the invention is homogenous and is substantially devoid of phase separation (or syneresis). The hydrogel is characterized by a greater viscosity than water (at RT, such as between 2° and 30° C.), usually at least 100, or at least 10000 cP.

In some embodiments, the hydrogel is characterized by a porosity of less than 50%, less than 40%, less than 30%, less than 20%, less than 10%, less than 5%, or between 5 and 50, between 5 and 30, between 5 and 10, between 5 and 20%, including any range between. In some embodiments, the porosity of the hydrogel is sufficient to support a diffusion of the active agent (secreted by the plurality of viable cells) from the inner portion of the hydrogel to the outer surface of the hydrogel, so that the active agent is available and can undergo uptake by the cells (e.g. the active agent is in contact with or in close proximity to the cells attached to the outer surface of the hydrogel).

In some embodiments, the hydrogel is characterized by a predefined pattern or shape. In some embodiments, the hydrogel in a form of a predefined pattern is bound to a support material. In some embodiments, the hydrogel is a printed material. In some embodiments, the hydrogel is in a form of particles.

In some embodiments, a water content of the composition (or of the hydrogel) is between 10% (w/w) and 90% (w/w), between 15% (w/w) and 90% (w/w), between 25% (w/w) and 90% (w/w), between 30% (w/w) and 90% (w/w), between 10% (w/w) and 80% (w/w), between 15% (w/w) and 80% (w/w), between 25% (w/w) and 80% (w/w), between 30% (w/w) and 80% (w/w), between 10% (w/w) and 75% (w/w), between 15% (w/w) and 75% (w/w), between 25% (w/w) and 75% (w/w), between 30% (w/w) and 75% (w/w), between 5% (w/w) and 60% (w/w), between 10% (w/w) and 60% (w/w), between 15% (w/w) and 60% (w/w), between 25% (w/w) and 60% (w/w), between 30% (w/w) and 60% (w/w), between 1% (w/w) and 50% (w/w), between 2% (w/w) and 50% (w/w), between 5% (w/w) and 50% (w/w), between 10% (w/w) and 50% (w/w), between 15% (w/w) and 50% (w/w), between 25% (w/w) and 50% (w/w), between 30% (w/w) and 50% (w/w), between 2% (w/w) and 40% (w/w), between 5% (w/w) and 40% (w/w), between 10% (w/w) and 40% (w/w), between 15% (w/w) and 40% (w/w), between 25% (w/w) and 40% (w/w), or between 30% (w/w) and 40% (w/w), including any range therebetween. Each possibility represents a separate embodiment of the present invention.

In some embodiments, the shape or pattern of the hydrogel and the crosslinking of the biopolymer is obtained by printing. In some embodiments, the shape or pattern of the hydrogel comprising the cross linked biopolymer is obtained by printing.

### Biopolymer

In some embodiments, the cross linkable biopolymer comprises (i) one or more of cross-linkable polysaccharide including any salt, any conjugate, or any hydrolysate thereof; (ii) one or more of cross-linkable protein, including any salt, any conjugate, or any hydrolysate thereof or both (i) and (ii).

In some embodiments, the cross linkable biopolymer comprises one or more of cross-linkable polysaccharide and a gelling agent. In some embodiments, the gelling agent is a gel-forming polymer (e.g. PVA; polyacrylates such as polyacrylic acid, or polyacrylic acid ester; a thermoresponsive polymer; a gel forming polysaccharide such as alginate, ulvan, chitosan, starch, modified starch, cellulose, modified cellulose such as alkylated or carboxyalkylated cellulose; a gel forming protein/peptide such gelatin, lamin, a cross-linkable protein, etc.). The gelling agent is configured to form a gel in an aqueous solution (e.g. spontaneously upon subjecting thereof to a trigger, such as temperature, or upon crosslinking thereof with a cross-linking agent). In some embodiments, the dry weight ratio of the gelling agent within the scaffold is between 40 and 80, between 50 and 75%, including any range between.

In some embodiments, the cross linkable biopolymer comprises a single cross-linkable polysaccharide specie and at least two cross-linkable protein species. In some embodiments, the cross linkable biopolymer within the scaffold of the invention consists essentially of a single cross-linkable polysaccharide specie and at least two cross-linkable protein species.

In some embodiments, the cross linkable biopolymer is in the form of self-assembled matrix within the scaffold (e.g. being in a form of hydrogel and/or particles disclosed herein). In some embodiments, the cross linkable biopolymer is capable of undergoing self-assembly in an aqueous solution (e.g. upon application of a trigger, such as heating, cooling, vibrations, mechanical impact, acoustic waves, etc.), thereby forming a three-dimensional polymeric network. In some embodiments, the cross linkable biopolymer is capable of forming a three-dimensional polymeric network upon crosslinking thereof with a crosslinking agent. In some embodiments, the three-dimensional polymeric network comprises at least partially cross-linked protein molecules and/or polysaccharide molecules. In some embodiments, the protein molecules and/or polysaccharide molecules within the three-dimensional polymeric network are cross-linked via non-covalent or via covalent interactions. In some embodiments, the three-dimensional polymeric network comprises intertwined polymers comprising protein molecules and/or polysaccharide molecules, and water molecules at least partially bound to the intertwined polymers. In some embodiments, the three-dimensional polymeric network and water molecules bound thereto form a hydrogel. In some embodiments, the cross linkable biopolymer is capable of forming a hydrogel in contact with an aqueous solution and optionally upon at least partial crosslinking thereof.

In some embodiments, the cross-linkable polysaccharide comprises one or more polysaccharides selected from alginic acid, hyaluronic acid, chitosan, dextran, agarose, a cellulose derivative (e.g. HPMC), modified starch, starch, a gum (such as locust bean gum, Guar gum, xanthan gum, gum Arabic, gellan gum) and carrageenan, including any salt, and co-polymer, or any combination thereof. In some embodiments, the cross-linkable polysaccharide is capable of forming hydrogel in an aqueous solution. In some embodiments, the cross-linkable polysaccharide is configured to undergo crosslinking upon contacting thereof with a crosslinking agent. In some embodiments, the cross-linkable polysaccharide (as used herein) is devoid of hyaluronic acid. Additional cross-linkable polysaccharides are well-known in the art.

In some embodiments, the cross-linkable protein is or comprises a structural protein (also termed as scleroprotein, or fibrous protein). Structural proteins are commonly constructed by elongated or fibrous polypeptide chains which form filamentous and sheet like structure. Scleroproteins typically have low solubility in water. In some embodiments, the structural proteins also promote cell attachment and proliferation.

In some embodiments, the cross-linkable protein comprises one or more proteins selected from gelatin, fibrinogen, collagen, vitronectin, laminin and fibronectin including any salt, any conjugate, or any hydrolysate thereof. In some embodiments, the cross-linkable protein is configured to undergo crosslinking upon contacting thereof with a crosslinking agent. Additional cross-linkable proteins are well-known in the art.

Collagen is the main structural protein in the extracellular matrix in the various connective tissues in the body of a mammal and consists of amino acids bound together to form a triple helix of elongated fibril (known as a collagen helix). Collagen is the most abundant protein in mammals, making up from 25% to 35% of the whole-body protein content. The hardness or rigidity of the collagen tissue depending upon the degree of mineralization, ranging from rigid (such as bones) to compliant (such as a tendon) or as a gradient covering the range from rigid to compliant (such as cartilage). The Collagen could be of type I-V and from various (mammalian) sources. Type I collagen is a component of skin, bone, tendon, and other fibrous connective tissues. In some embodiments, the collagen is type I collagen.

Gelatin (also referred to as gelatine, hydrolyzed collagen, collagen hydrolysate, gelatine hydrolysate and hydrolyzed gelatine) is a translucent, colorless and flavorless material often used as a food ingredient. Gelatin is a heterogeneous mixture of water-soluble proteins of high average molecular masses, present in collagen. The proteins are extracted by boiling skin, tendons, ligaments, bones, etc. in water. Gelatin is derived from collagen that has been partially purified and hydrolyzed. Collagen hydrolysis is performed by one of three different methods: acid-, alkali-, and enzymatic hydrolysis. Gelatin is brittle when dry and gummy when moist. Gelatin forms thermally reversible gels with water and can form stable foams.

In some embodiments, the collagen and gelatin are characterized by at least one of: 95-99% protein by dry weight, amino acid content being 20-30%, or 24-25% proline and/or hydroxyproline, 20-21% glycine, 10-11% glutamic acid, 8% arginine, 8-9% alanine and 28% other amino acids.

Vitronectin (VTN or VN) is a glycoprotein of the hemopexin family which is abundantly found in serum, the extracellular matrix and bone.

Laminins are high-molecular weight (−400 to −900 kDa) glycoproteins of the extracellular matrix. They are a major component of the basal lamina (one of the layers of the basement membrane), a protein network foundation for most cells and organs. Laminins are heterotrimeric proteins that contain an a-chain, a P-chain, and a y-chain (often found in five, four, and three genetic variants, respectively). The trimeric proteins intersect to form a cross-like structure that can bind to other cell membrane and extracellular matrix molecules.

Fibronectin is a high-molecular weight (−440 kDa) glycoprotein component of the extracellular matrix that binds to membrane-spanning receptor proteins called integrins, in addition to other extracellular matrix proteins such as collagen, fibrin, and heparan sulfateproteoglycans (e.g. syndecans). Two types of fibronectin are present in vertebrates: soluble plasma fibronectin (a major protein component of blood plasma) and insoluble cellular fibronectin (a major component of the extracellular matrix).

Fibrinogen is a large, fibrous, non-globular and soluble glycoprotein that is involved in blood clotting, as thrombin converts it into insoluble fibrin molecule, in the presence of Ca2+ via intermolecular interactions. Thrombin converts fibrinogen to fibrin and then to a fibrin-based blood clot. Fibrin clots function primarily to occlude blood vessels to stop bleeding. The scaffold of the invention in some embodiments thereof encompasses gelatin, crosslinked alginate and collagen crosslinked by thrombin and Fibrinogen. Since upon sole addition of fibrinogen to collagen no substantial collagen crosslinking occurs (prior to addition of thrombin), fibrinogen is referred to herein as the “cross-linkable protein”, and as the “crosslinking agent”.

The fibrinogen protein is a 45 nm hexameric arrangement of peptide chains (alpha, beta, and gamma) connected by disulfide bonds, with a molecular weight of approximately 340 kDa. The fibrinogen protein consists of two outer D domains and a central E domain and is encoded by chromosome 4. Fibrin can form a fibrin scaffold as a network of protein that holds together and supports a variety of living tissues. It is produced naturally by the body after injury, but also can be engineered as a tissue substitute to speed healing.

The term “hydrolysate” encompasses fragments such as peptides (derived from a native or non-hydrolyzed protein), or poly-/oligo-saccharides (derived from a native or non-hydrolyzed polysaccharide). The average MW of the fragments (e.g. peptides or poly-/oligo-saccharides) may vary being in general between 1000 and 100000 Da, between 1000 and 50000 Da, 1000 and 10000 Da, 10000 and 100000 Da, including any range between. The term “hydrolysate” encompasses fragments obtained via a chemical hydrolysis (e.g. by an acid or base) or via an enzymatic hydrolysis of a native polysaccharide or native protein. The native polysaccharide or native protein encompass natural polymers, extracted natural polymers, and/or synthetic polymers.

In some embodiments, the term “derived from” encompasses any industrial processing such as purification, isolation, fractionation, chemical modification, etc.

The term “conjugate” encompasses any chimeric molecule containing the cross linkable biopolymer covalently bound to another molecule (e.g. a small molecule, an oligomer, or a polymer) which is not the cross linkable biopolymer. The term “conjugate” further encompasses a co-polymer (e.g. graft copolymer, block-copolymer, random copolymer, etc.), wherein the co-polymer comprises at least two different polymeric species (optionally 2 different cross linkable biopolymers, or a cross linkable biopolymer and an additional polymer which is not the cross linkable biopolymer).

In some embodiments, the alginate is characterized by an average molecular weight (MW) between 10,000 and 1,000,000 Da, between 10,000 and 100,000 Da, between 100,000 and 1,000,000 Da, between 100,000 and 10,000,000 Da, between 500,000 and 10,000,000 Da, between 100,000 and 500,000 Da, including ant range between. The terms “alginate” and “alginic acid” are used herein interchangeably and encompass the polysaccharide in both deprotonated and protonated state (i.e. having carboxyl and/or carboxylate groups) and further encompass any salt thereof.

In some embodiments, the scaffold of the invention comprises the cross linkable biopolymer in a form of a matrix, and a plurality of viable cells encapsulated or embedded within the cross linkable biopolymer; wherein the cross linkable biopolymer consists essentially of (i) one or more of the cross-linkable polysaccharides selected from alginic acid, chitosan, a gum, dextran, agarose, and carrageenan including any salt, and copolymer, or any conjugate thereof, and (ii) plurality of cross linkable proteins selected from gelatin, collagen, fibrinogen, vitronectin, laminin and fibronectin including any salt, any conjugate, or any hydrolysate thereof. In some embodiments, plurality of cross linkable proteins comprises gelatin, collagen and fibrinogen.

In some embodiments, the scaffold of the invention comprises the cross linkable biopolymer in a form of a matrix. In some embodiments, the scaffold of the invention comprises the cross linkable biopolymer in a form of a matrix, and a plurality of viable cells encapsulated or embedded within the matrix; wherein the cross linkable biopolymer consists essentially of (i) alginic acid, including any salt, and copolymer, or any conjugate thereof, and (ii) gelatin, collagen and fibrinogen, including any salt, any conjugate, or any hydrolysate thereof. In some embodiments, at least 60%, at least 70%, at least 80%, at least 90%, at least 95%, or between 60 and 95%, between 70 and 95%, between 80 and 95%, between 80 and 90%, between 80 and 97%, or between 80 and 99% by dry weight of the scaffold (not including the first and second cell population) consist of (i) alginic acid, including any salt, and copolymer, or any conjugate thereof, and (ii) gelatin, collagen (e.g. collagen type I) and fibrinogen, including any salt, any conjugate, or any hydrolysate thereof; and (iii) the crosslinking agent, including any range between. In some embodiments, at least 60%, at least 70%, at least 80%, at least 90%, at least 95%, or between 60 and 95%, between 70 and 95%, between 80 and 95%, between 80 and 90%, between 80 and 97%, or between 80 and 99% by dry weight of the scaffold (not including the first and second cell population) consist of (i) alginic acid, including any salt, and copolymer, or any conjugate thereof, (ii) the gelling agent and (iii) the crosslinking agent, including any range between.

In some embodiments, at least 60%, at least 70%, at least 80%, at least 90%, at least 95%, or between 60 and 95%, between 70 and 95%, between 80 and 95%, between 80 and 90%, between 80 and 97%, or between 80 and 99% by dry weight of the scaffold (not including the first and second cell population) consist of (i) crosslinked alginic acid, including any salt, and copolymer, or any conjugate thereof, (ii) crosslinked collagen and (iii) the gelling agent (e.g. gelatin), including any range between.

In some embodiments, at least 60%, at least 70%, at least 80%, at least 90%, at least 95%, or between 60 and 95%, between 70 and 95%, between 80 and 95%, between 80 and 90%, between 80 and 97%, or between 80 and 99% by dry weight of the scaffold (not including the first and second cell population) consist of (i) alginic acid, including any salt, and copolymer, or any conjugate thereof, (ii) the gelling agent, (iii) collagen and (iv) the crosslinking agent, including any range between.

In some embodiments, a w/w ratio between the cross linkable protein and the cross linkable polysaccharide within the composition (e.g. the scaffold) of the invention is between about 3:1 and about 1:3, between about 3:1 and about 2:1, between about 3:1 and about 1:1, between about 1:1 and about 1:3, including any range between. In some embodiments, a w/w ratio between the cross linkable protein and cross linkable polysaccharide within the composition (e.g. the scaffold) of the invention is between about 3:1 and about 2:3, or between about 3:1 and 1:1, including any range between.

In some embodiments, the cross linkable protein is or comprises collagen and gelatin, and the cross linkable polysaccharide is alginate, wherein a w/w ratio between the cross linkable protein and alginate within the scaffold of the invention is between about 3:1 and about 2:3, or between about 3:1 and 1:1, including any range between.

In some embodiments, a crosslinking agent is selected from thrombin, a multivalent metal cation, a polyanion, a cross-linking enzyme (such as tyrosinase, peroxidase, transglutaminase, etc.), and a chemical cross-linker, including any salt and any combination thereof.

Crosslinking agent or factor, refers to a compound (or compounds) that induces crosslinking of the cross linkable biopolymer. In some embodiments, crosslinking is generated in a rapid manner at room temperature (e.g. between 20 and 35 C), for example within a time period ranging up to 10, up to 5 or up to 1 minute.

In some embodiments, the scaffold comprises more than one crosslinking agent specie. In some embodiments, the crosslinking agent comprises a coagulating agent (such as cerastocytin, thrombin, etc.)

Thrombine is a serine protease that converts soluble fibrinogen into insoluble strands of fibrin. The individual fibrin strands aggregate to form a three-dimensional gel-like structure by polymerizing and crosslinking with other fibrin stands. Fibrin regulates the aggregation by possessing three low affinity binding sites (two in fibrin's E domain; one in the D domain) for thrombin; this binding sequesters thrombin from attacking fibrinogen. The processes happen in the presence of divalent metal cations (such as calcium).

In some embodiments, the multivalent metal cation comprises a divalent metal cation (e.g. Pb²⁺, Cu²⁺, Cd²⁺, Ba²⁺, Ca²⁺, Sr²⁺, Co²⁺, Ni²⁺, Mg²⁺, Zn²⁺ or Mn²⁺), or a trivalent metal cation (e.g. Al, Fe cation). In some embodiments, the divalent metal cation comprises an alkaline earth metal cation (e.g. Ba²⁺, Ca²⁺, Sr²⁺ cation including any combination thereof). In some embodiments, the crosslinking agent comprises a salt of the multivalent metal cation (e.g. a halide salt, a carbonate salt, etc.)

In some embodiments, a multivalent metal cation comprises Ca2+. In some embodiments, the crosslinking agent comprises a divalent calcium salt.

In some embodiments, the alginic acid is at least partially crosslinked within the composition. In some embodiments, the alginic acid is at least partially crosslinked by Ca(II) cations. In some embodiments, a w/w portion of the crosslinking agent (e.g. a multivalent cation, such as Ca(II) cation) relative to the cross-linkable polysaccharide (e.g. alginic acid) within the scaffold is between about 1 and about 20%, between about 5 and about 20%, between about 1 and about 10%, between about 5 and about 15%, between about 8 and about 12%, including any value or any range between.

In some embodiments, a w/w portion of Ca(II) cations relative to the alginic acid within the scaffold is between about 1 and about 20%, between about 5 and about 20%, between about 1 and about 10%, between about 5 and about 15%, between about 8 and about 12%, including any value or any range between.

In some embodiments, the collagen is at least partially crosslinked within the composition. In some embodiments, the collagen is crosslinked by fibrinogen and thrombin. Exemplary composition of the gel and a method for preparation thereof are provided in the Examples section.

In some embodiments, a w/w ratio between collagen and fibrinogen within the scaffold is between about 2:1 and 1:2, between about 1.5:1 and 1:1.5, about 1:1, or any range between. In some embodiments, a molar ratio between collagen and fibrinogen within the scaffold is between about 2:1 and 1:2, between about 1.5:1 and 1:1.5, about 1:1, or any range between. In some embodiments, a molar ratio between collagen and fibrinogen within the scaffold is about 1:1.

In some embodiments, a w/w ratio between alginic acid and gelatin within the scaffold is between about 2:1 and 1:5, between about 1:1 and 1:5, between about 1:1 and 1:3, between about 1:2 and 1:3, or between about 1:1.5 and 1:3, including any value or any range between.

In some embodiments, a w/w ratio between collagen and alginic acid within the scaffold is between about 1:10 and 1:100, between about 1:20 and 1:100, between about 1:30 and 1:100, between about 1:30 and 1:80, or between about 1:50 and 1:80, including any range between.

In some embodiments, a combined weight portion of any one of alginic acid and gelatin by dry weight of the composition (or by dry weight of the scaffold) is between about 50 and 90% w/w, between about 60 and 95% w/w, between about 50 and 95% w/w, including any range between.

In some embodiments, a w/w concentration of any one of alginate and fibrinogen by dry weight of the composition (or by dry weight of the scaffold) is between about 0.5 and about 5% w/w, between about 1 and about 5% w/w, between about 1 and about 10% w/w, including any range between.

In some embodiments, a combined weight portion of alginic acid and gelatin relative to the total weight of the cross-linkable biopolymer is between about 50 and 90% w/w, between about 60 and 95% w/w, between about 50 and 95% w/w, including any range between.

In some embodiments, a combined weight portion of fibrinogen and gelatin by dry weight of the composition (or by dry weight of the scaffold) is between about 50 and 90% w/w, between about 60 and 95% w/w, between about 50 and 95% w/w, including any range between.

In some embodiments, at least 60%, at least 70%, at least 80%, at least 90%, at least 95%, or between 60 and 95%, between 70 and 95%, between 80 and 95%, between 80 and 90%, between 80 and 97%, or between 80 and 99% by dry weight of the scaffold (not including the first cell population) consist of (i) alginic acid, including any salt, and copolymer, or any conjugate thereof, and (ii) the cross-linkable protein comprising gelatin and collagen, including any salt, any conjugate, or any hydrolysate thereof; and (iii) the crosslinking agent comprising fibrinogen, thrombin, and Ca2+ including any range between.

In some embodiments, at least 60%, at least 70%, at least 80%, at least 90%, at least 95%, or between 60 and 95%, between 70 and 95%, between 80 and 95%, between 80 and 90%, between 80 and 97%, or between 80 and 99% by dry weight of the scaffold (not including the first cell population) consist of (i) alginic acid, including any salt, and copolymer, or any conjugate thereof, and (ii) the cross-linkable protein comprising gelatin and collagen, including any salt, any conjugate, or any hydrolysate thereof; and (iii) the crosslinking agent comprising fibrinogen, thrombin, and Ca2+ including any range between; and wherein at least one of (i) to (v) or any combination thereof:
(i) a weight portion of the cross-linkable protein relative to the dry weight of the scaffold constituents is between 50 and 80%;
(ii) a weight portion of the alginate relative to the dry weight of the scaffold constituents is between 5 and 20%;
(iii) a weight ratio between the cross-linkable protein and the cross-linkable polysaccharide in the scaffold is between 3:1 and 1:1;
(iv) a weight portion of the cross-linkable protein relative to the dry weight of the scaffold constituents is between 50 and 80%;
(v) a weight portion of Ca2+ relative to alginate is between 1 and 20%.

In some embodiments, the gel content (also used as “gelation content”) of the scaffold is between 20 and 60%, between 30 and 60%, between 40 and 60%, between 40 and 70%, including any range between.

### Functional Moiety

In some embodiments, the functional moiety is a polyamino acid. In some embodiments, the functional moiety is or comprises a small molecule recognizable by the target (e.g. cell receptor, such as CD44). In some embodiments, the functional moiety is or comprises a natural ligand of a cell receptor. In some embodiments, the functional moiety is or comprises a natural ligand of a cell surface receptor. In some embodiments, the natural ligand is a small molecule (e.g. a natural compound). In some embodiments, the functional moiety binds a target on the cell membrane. In some embodiments, the functional moiety binds CD44 receptor on the cell membrane. In some embodiments, the functional moiety binds an extracellular target on the cell membrane. In some embodiments, the functional moiety comprises a single specie or a plurality of chemically distinct species. The terms “functional moiety” and “ligand” are used herein interchangeably.

In some embodiments, the functional moiety hybridizes to its target. In some embodiments, the functional moiety is complementary to its target. In some embodiments, the functional moiety is an antibody or antigen binding fragment thereof. The structure of antibodies is well known and though a skilled artisan may not know to what target an antibody binds merely by its CDR sequences, the general structure of an antibody and its antigen binding region can be recognized by a skilled artisan.

As used herein, the term “antibody” refers to a polypeptide or group of polypeptides that include at least one binding domain that is formed from the folding of polypeptide chains having three-dimensional binding spaces with internal surface shapes and charge distributions complementary to the features of an antigenic determinant of an antigen. An antibody typically has a tetrameric form, comprising two identical pairs of polypeptide chains, each pair having one “light” and one “heavy” chain. The variable regions of each light/heavy chain pair form an antibody binding site. An antibody may be oligoclonal, polyclonal, monoclonal, chimeric, camelised, CDR-grafted, multi-specific, bi-specific, catalytic, humanized, fully human, anti-idiotypic and antibodies that can be labeled in soluble or bound form as well as fragments, including epitope-binding fragments, variants or derivatives thereof, either alone or in combination with other amino acid sequences. An antibody may be from any species. The term antibody also includes binding fragments, including, but not limited to Fv, Fab, Fab′, F(ab′)2 single stranded antibody (svFC), dimeric variable region (Diabody) and disulphide-linked variable region (dsFv). In particular, antibodies include immunoglobulin molecules and immunologically active fragments of immunoglobulin molecules, i.e., molecules that contain an antigen binding site. Antibody fragments may or may not be fused to another immunoglobulin domain including but not limited to, an Fc region or fragment thereof. The skilled artisan will further appreciate that other fusion products may be generated including but not limited to, scFv-Fc fusions, variable region (e.g., VL and VH)˜Fc fusions and scFv-scFv-Fc fusions.

Immunoglobulin molecules can be of any type (e.g., IgG, IgE, IgM, IgD, IgA and IgY), class (e.g., IgG1, IgG2, IgG3, IgG4, IgAQ1 and IgA2) or subclass.

In some embodiments, the antibody comprises a heavy chain and a light chain. In some embodiments, the antibody is a heavy chain only antibody. In some embodiments, the antibody is an antibody mimetic.

As used herein, the terms “peptide”, “polypeptide”, “polyamino acid” and “protein” are used interchangeably to refer to a polymer of amino acid residues. In another embodiment, the terms “peptide”, “polypeptide” and “protein” as used herein encompass native peptides, peptidomimetics (typically including non-peptide bonds or other synthetic modifications) and the peptide analogues peptoids and semipeptoids or any combination thereof. In another embodiment, the peptides polypeptides and proteins described have modifications rendering them more stable while in the body or more capable of penetrating into cells. In one embodiment, the terms “peptide”, “polypeptide”, “polyamino acid” and “protein” apply to naturally occurring amino acid polymers including or consisting essentially of 21 naturally occurring amino acids. In one embodiment, the terms “peptide”, “polypeptide”, “polyamino acid” and “protein” apply to naturally occurring amino acid polymers including or consisting essentially of 21 naturally occurring amino acid residues bound to each other via alpha peptide bonds. In one embodiment, the terms “peptide”, “polypeptide”, “polyamino acid” and “protein” apply to naturally occurring amino acid polymers including or consisting essentially of 21 naturally occurring amino acid residues bound to each other via a primary amide bond. In one embodiment, the terms “peptide”, “polypeptide”, “polyamino acid” and “protein” apply to naturally occurring amino acid polymers including or consisting essentially of 21 naturally occurring amino acid residues bound to each other via a peptide bond formed by a formal condensation between alpha amino group of the first amino acid and alpha carboxy group of the next following amino acid. In one embodiment, the terms “peptide”, “polypeptide”, “polyamino acid” and “protein” apply to naturally occurring amino acid polymers including or consisting essentially of 21 naturally occurring amino acid residues bound to each other via a peptide bond formed by a formal condensation between (i) alpha amino group, or alpha carboxy group of the first amino acid, and (ii) a side chain amino group, or a side chain carboxy group of the next following amino acid. In one embodiment, the terms “peptide”, “polypeptide”, “polyamino acid” and “protein” apply to naturally occurring amino acid polymers including or consisting essentially of 21 naturally occurring amino acids. In another embodiment, the terms “peptide”, “polypeptide”, “polyamino acid” and “protein” apply to amino acid polymers in which one or more amino acid residue is an artificial chemical analogue of a corresponding naturally occurring amino acid.

The term “artificial chemical analogue” or “chemical derivative” includes any chemical derivative of the polypeptide having one or more residues chemically derivatized by reaction on the side chain or on any functional group within the peptide. Such derivatized molecules include, for example, peptides bearing one or more protecting groups (e.g., side chain protecting group(s) and/or N-terminus protecting groups), and/or peptides in which free amino groups have been derivatized to form amine hydrochlorides, p-toluene sulfonyl groups, carbobenzoxy groups, t-butyloxycarbonyl groups, acetyl groups or formyl groups. Free carboxyl groups may be derivatized to form amides thereof, salts, methyl and ethyl esters or other types of esters or hydrazides. Free hydroxyl groups may be derivatized to form O-acyl or O-alkyl derivatives. The imidazole nitrogen of histidine may be derivatized to form N-im-benzylhistidine. Also included as chemical derivatives are those peptides, which contain one or more naturally occurring amino acid derivatives of the twenty standard amino acid residues. For example: 4-hydroxyproline may be substituted for proline; 5-hydroxylysine may be substituted for lysine; 3-methylhistidine may be substituted for histidine; homoserine may be substituted or serine; and Dab, Daa, and/or ornithine (O) may be substituted for lysine.

In some embodiments, the functional moiety comprises hyaluronic acid (HA). In some embodiments, the HA is characterized by an average molecular weight (MW) between 10,000 and 1,000,000 Da, between 10,000 and 100,000 Da, between 100,000 and 1,000,000 Da, between 100,000 and 10,000,000 Da, between 500,000 and 10,000,000 Da, between 100,000 and 500,000 Da, including ant range between. The term “hyaluronic acid” encompasses the polysaccharide in both deprotonated and protonated state (i.e. having carboxyl and/or carboxylate groups) and further encompasses any salt thereof.

In some embodiments, provided herein the scaffold of the invention consisting essentially of the cross-linkable biopolymer and a plurality of viable cell; wherein at least 60%, at least 70%, at least 80%, at least 90%, at least 95%, or between 60 and 95%, between 70 and 95%, between 80 and 95%, between 80 and 90%, between 80 and 97%, or between 80 and 99% including any range between, by dry weight of the scaffold consists of: (i) alginic acid, including any salt, and copolymer, or any conjugate thereof, and (ii) the cross-linkable protein comprising gelatin, collagen and fibrinogen, including any salt, any conjugate, or any hydrolysate thereof; and (iii) the crosslinking agent consisting of fibrinogen, thrombin, and Ca²⁺; and wherein the scaffold is covalently bound to the functional moiety comprising hyaluronic acid.

In some embodiments, a dry weight ratio between the functional moiety and the cross-linkable biopolymer is between 0.0001:1 and 1:1, between 0.001:1 and 1:1, between 0.01:1 and 1:2, between 0.1:1 and 1:1, between 0.001:1 and 0.1:1, including any range between.

In some embodiments, a weight ratio between the functional moiety and the hydrogel is between 1 ng and 1 mg, or between 1 ng and 1 ug of the functional moiety relative to 10-20 mg of the hydrogel, including any range between.

In some embodiments, the functional moiety is bound to the scaffold (e.g. hydrogel, or microparticle) via a covalent bond. In some embodiments, the functional moiety is bound to the scaffold via an amide bond, via an ester bond, a linker, a click reaction product, a thioether bond, a linker, or any combination thereof. The linker is not part of the functional moiety and is also not a part of the microparticle (e.g. of the cross linkable biopolymer), but rather is attached (conjugated) to each one and thereby links them.

In some embodiments, the linker is substantially stable in a cell culture (e.g. within a cell culture medium) or in a tissue for at least 1, 2, 3, 4, 5, 6, 7, 8, 9, 10, 11, 12, 18, 24, 36, 48, 72 hours, 5 days, 10 days or more, including any range between.

In some embodiments, the linker is or comprises a linear or a branched chain. In some embodiments, the linker of the invention is or comprises a backbone optionally comprising one or more the chain.

In some embodiments, the linker is a spacer (e.g., a natural and/or unnatural amino acid, alkyl, an amide bond, an ester bond, a thioester bond, a urea bond, including any derivative or a combination thereof). In some embodiments, the linker comprises a biocompatible polymer or a biocompatible moiety. In some embodiments, the biocompatible polymer is at least partially biodegradable. In some embodiments, the biocompatible polymer is or comprises a polyglycol ether, a polyester, a polyamide, a polyamino acid, a peptide and/or a derivative thereof or any combination thereof. In some embodiments, the polyglycol ether is or comprises polyethylene glycol (PEG).

In some embodiments, the linker further comprises a spacer (e.g., a natural and/or unnatural amino acid, alkyl, an amide bond, an ester bond, a disulfide bond, a thioester bond, a urea bond, including any derivative or a combination thereof).

In some embodiments, the linker further comprises a click reaction product (e.g., a covalent linkage such as a cyclization reaction product, and/or a succinimide-thioether moiety formed via a click reaction).

Click reactions are well-known in the art and comprise inter alia Michael addition of maleimide and thiol (resulting in the formation of a thioether bond); azide alkyne cycloaddition; Diels-Alder reaction (e.g., direct and/or inverse electron demand Diels Alder); dibenzyl cyclooctyne 1,3-nitrone (or azide) cycloaddition; alkene tetrazole photoclick reaction etc.

In some embodiments, the scaffold has a predefined shape and is characterized by an inner portion facing or in contact with the plurality of viable cells; and an outer portion facing an ambient. In some embodiments, the functional moiety is bound to at least a portion of the outer surface of the scaffold.

In some embodiments, the functional moiety is bound to at least a portion of the outer surface of the scaffold. In some embodiments, at least 0.01%, at least 0.1%, at least 1%, at least 10% or between 1 and 10%, between 1 and 30%, or 1 and 50% of the outer surface of the scaffold is in contact with or covered by the functional moiety, including any range between.

In some embodiments, the functional moiety is bound to at least one constituent of the scaffold. In some embodiments, the functional moiety is bound to at least one cross linkable biopolymer. In some embodiments, the functional moiety is bound to at least one cross linkable polysaccharide. In some embodiments, the functional moiety is bound to at least one cross linkable protein. In some embodiments, the functional moiety is bound to at least one cross linkable protein and to at least one cross linkable polysaccharide. In some embodiments, the functional moiety is bound to alginate via an ester bond. In some embodiments, at least one carboxy group of the functional moiety is bound to a hydroxy group of alginate and/or gelatin, or vice versa (i.e. the functional moiety is bound to the scaffold via an ester bond).

In some embodiments, at least one carboxy group of the functional moiety is bound to an amino group of the cross linkable protein (i.e. the functional moiety is bound to the scaffold via an amide bond).

In some embodiments, at least 60%, at least 70%, at least 80%, at least 90%, at least 95%, or between 60 and 95%, between 70 and 95%, between 80 and 95%, between 80 and 90%, between 80 and 97%, or between 80 and 99% by dry weight of the scaffold (not including the first/second cell population) consist of alginic acid, including any salt, and copolymer, or any conjugate thereof, wherein the cross-linkable protein comprising gelatin, collagen and fibrinogen, including any salt, any conjugate, or any hydrolysate thereof; wherein the crosslinking agent comprising fibrinogen, thrombin, and Ca²⁺ including any range between; wherein the scaffold is covalently bound to HA (e.g. via the hydroxy group of the cross-linkable polysaccharide or via amino group of the cross linkable protein), wherein the amount of HA is between 1 ng and 1 μg, or between 1 ng and 1 mg per 10 mg of the scaffold (i.e. a hydrogel scaffold) and wherein at least one of (i) to (v) or any combination thereof:
(i) a weight portion of the cross-linkable protein relative to the dry weight of the scaffold constituents is between 50 and 80%;
(ii) a weight portion of the alginate relative to the dry weight of the scaffold constituents is between 5 and 20%;
(iii) a weight ratio between the cross-linkable protein and the cross-linkable polysaccharide in the scaffold is between 3:1 and 1:1;
(iv) a weight portion of the cross-linkable protein relative to the dry weight of the scaffold constituents is between 50 and 80%;
(v) a weight portion of Ca2+ relative to alginate is between 1 and 20%.

### Cells

In some embodiments, the plurality of viable cells is embedded into the scaffold. In some embodiments, the plurality of viable cells are encapsulated inside the scaffold (i.e. within the inner portion of the scaffold). In some embodiments, the outer surface of the scaffold is substantially devoid of the plurality of viable cells. In some embodiments, between 80 and 100%, between 80 and 90%, between 80 and 95%, between 80 and 97%, between 80 and 99% of the total cell number of the plurality of viable cells are located within the inner portion of the scaffold.

In some embodiments, the number of viable cells within the scaffold of the invention is predetermined by the volume and/or at last one dimension of the crosslinked cell scaffold. In some embodiments, the number of viable cells is further predetermined by the cell type, cell dimension, proliferation rate, etc.

A skilled artisan will appreciate that different cell types may have different maximum threshold concentration within the crosslinked cell scaffold due to various factors predetermining cell viability within the scaffold (such as cell dimension, proliferation rate, etc.). The maximum threshold concentration is so as to provide appropriate conditions suitable for cell viability and for substantially prevent cell mortality, resulting in a composition of the invention wherein at least 50%, at least 70%, at least 80%, at least 85%, at least 90%, at least 95% of the cells are live cells, including any range between. Furthermore, a skilled artisan will appreciate that there is no minimum amount of the viable cells, however it is presumed that the minimum amount is predetermined by the amount of cells required for an efficient production and secretion of the active agent. Thus, the minimum/maximum threshold of the viable cells within the crosslinked cell scaffold can be easily determined based on conventional methods known in the art.

In some embodiments, the plurality of viable cells are configured to produce and/or to secret an active agent. The term “viable cells” encompass live cells. The term “live cell” as used herein encompasses a cell performing one or more of replicating a genome or DNA, cell proliferation or replication, RNA synthesis, protein translation, energy production process, fermentation or any equivalent energy production process, secretion of an active compounds (e.g. metabolite or a cell metabolite, or the active agent disclosed herein).

In some embodiments, the viable cells are viable within the composition of the invention, and/or upon application of the composition of the invention for cell growth, and/or upon contacting the composition of the invention with an aqueous cell medium.

In some embodiments, the plurality of viable cells comprises artificial cells. In some embodiments, the plurality of viable cells comprises mammalian cells. In some embodiments, the plurality of viable cells are in a form of a cell culture, a tissue, or both. In some embodiments, the plurality of viable cells comprises a single cell type or a plurality of cell types (e.g. co-culture).

In some embodiments, the plurality of viable cells is configured to produce and/or secrete an active agent in a cell culture. In some embodiments, the plurality of viable cells is capable configured to synthesize (i.e. to produce) the active agent. In some embodiments, the term “secreting” refers to the ability of the cell to provide (e.g. via a passive or active transport) the active agent outside the cell (e.g. into the surrounding inner portion of the scaffold, or into an aqueous solution such as a cell culture medium).

In some embodiments, the plurality of viable cells are mammalian cells configured to produce and/or to secret the active agent.

According to another aspect of the invention, there is provided a composition comprising the scaffold in a form of a hydrogel covalently bound to a functional moiety, wherein the hydrogel comprises the cross linkable biopolymer and a first cell configured produce and/or to secret an active agent; the cross linkable biopolymer is at least partially crosslinked by the crosslinking agent within the hydrogel; and the functional moiety has a binding affinity to a second cell. In some embodiments, the first cell and the second cell comprises a plurality of cells, also used herein as “first cell population” and “second cell population”, respectively. In some embodiments, each of the first cell population and of the second cell population consists of a single cell type or of distinct cell types.

In some embodiments, the first cell population and second cell population are mammalian cells. In some embodiments, the first cell population is configured to secret the active agent. In some embodiments, the first cell population is compatible with the hydrogel (i.e. the first cell population is viable within the hydrogel). In some embodiments, the first cell population is configured to secrete a growth factor in an cell culture, so as to obtain a concentration of the growth factor within an aqueous cell culture medium of between 100 and 1000 ng/ml, between 200 and 1000 ng ng/ml, between 200 and 500 ng/ml, between 200 and 700 ng/ml, including any range between.

In some embodiments, the active agent is essential for activity of the second cell population. In some embodiments, activity of the second cell population encompasses viability, proliferation, differentiation, cell growth, biochemical activity, or any combination thereof. In some embodiments, the second cell population comprises cultured cells (such as cells utilized for the production of the cultured meat), or of a tissue.

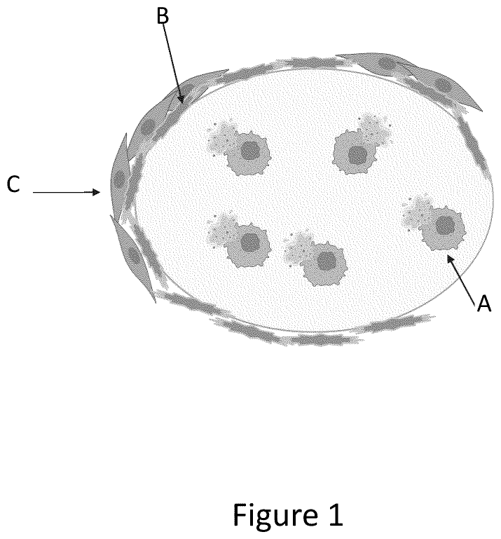

In some embodiments, the composition of the invention is for use in the delivering or supplementing the active agent to the second cell population, wherein the second cell population is in a form of a cell culture, a tissue, or both. In some embodiments, the composition of the invention is for use in the stimulating of cell growth, cell differentiation and/or cell proliferation of the second cell population. The composition of invention comprising the first cell population incorporated into the scaffold (e.g. in a form of a bead-like particle) and the second cell populations attached to the outer surface of the scaffold via HA is schematically illustrated in FIG. 1.

The terms “first cell population” and “plurality of viable cells” are used herein interchangeably. The terms “second cell population” and “cell(s)” are used herein interchangeably.

In some embodiments, the first cell population comprises or consist essentially of mammalian cells configured to produce and/or to secret the active agent. In some embodiments, the first cell population comprises or consist essentially of mammalian cells configured to produce and/or to secret a growth factor (e.g. Transforming growth factor beta (TGF-β)). In some embodiments, the first cell population comprises or consist essentially of TGF-β secreting cells. In some embodiments, the first cell population comprises or consist essentially of any one of a macrophage (differentiated or not), a colon epithelial cell, a lung epithelial cells, and a monocyte, including any combination thereof.

In some embodiments, a concentration of the first cell population within the scaffold is between 1000 and 200000, between 10000 and 200000, between 10000 and 50000, between 50000 and 200000 cell units per 10 mg of the scaffold (i.e. hydrogel), including any range between.

In some embodiments, a concentration of the second cell population within the scaffold is between 500 and 2000000, between 10000 and 2000000, between 50000 and 2000000, between 100000 and 2000000 cell units per 10 mg of the scaffold (i.e. hydrogel), including any range between.

In some embodiments, the active agent is a cell nutrient. In some embodiments, the active agent is a signaling molecule. In some embodiments, the active agent is a hormone. In some embodiments, the active agent is configured to modify (e.g. enhance or decrease) activity of the cells. In some embodiments, the active agent is configured to modify cell proliferation, cell differentiation, etc. In some embodiments, the active agent is configured to modify cell proliferation, and/or cell differentiation, of the second cell population.

In some embodiments, the active agent comprises a small molecule (e.g. a signaling molecule), a growth factor, a differentiation factor, a polyamino acid (such as an antibody, enzyme, peptide, etc.), a polynucleic acid, or any combination, thereof. In some embodiments, the small molecule encompasses any organic molecule having a MW below 1000 Da, or below 500 Da.

In some embodiments, the active agent is or comprises one or more growth factor(s), and/or differentiation factor(s). In some embodiments, the growth factor comprises any one of Transforming growth factor beta; Insulin-like growth factor 1; fibroblast growth factor2; epidermal growth factor; interleukin 6; Neuregulin 1, or any combination thereof.

In some embodiments, the second cell population comprises mammalian cells selected from any one of: Embryonic stem cells, Induced pluripotent stem cells, mesenchymal stem cells, Fibro-adipogenic progenitor cells, Muscle satellite cells, Fibroblasts, Endothelial cell, and Smooth muscle cells, including any combination thereof.

### Particles

In some embodiments, the scaffold of the invention is characterized by a predefined shape or is in a form of a predefined pattern in contact with a support material. In some embodiments, the functional moiety (e.g. hyaluronic acid, including any salt or a conjugate/copolymer thereof) is bound to at least a portion of the outer surface of the predefined pattern or of the predefined shape. In some embodiments, the support material is a solid. In some embodiments, the support material comprises any of a polymeric material (plastic), a metallic material, a glass material, a composite material, and wherein the support material is compatible with conditions suitable for cell culture (e.g. aqueous cell culture medium, a temperature of about 36 C, etc.).

In some embodiments, the composition of the invention is in a form of particles. In some embodiments, the scaffold is in a form of particles. In some embodiments, the hydrogel is in a form of particles. In some embodiments, the particles are gel particles. In some embodiments, the composition is a stable semisolid (or gelled) composition. In some embodiments, the composition is stable for at least 6 hours (h), at least 12 h, at least 24 h, at least 48 h, at least 72 h, at least 96 h, at least 10 days (d), at least one month (m), at least 6 m, at least 12 m, including any range therebetween.

As used herein the term “stable” (in the context of semisolid/gelled) particle composition), refers to the ability of the composition to maintain substantially its intactness, such as being substantially devoid of aggregation. In some embodiments, aggregates refer to a plurality of particles adhered or bound to each other. In some embodiments, a stable composition is substantially devoid of free (e.g., non-encapsulated) viable cells. In some embodiments, the composition of the invention is referred to as stable when at least 70%, at least 80%, at least 85%, at least 90%, at least 95%, at least 97% including any range between, of the initial cell loading remains bound to or encapsulated within the crosslinked cell scaffold under suitable storage conditions and for a time period described herein.

In some embodiments, the composition of the invention is referred to as stable, when at least 70%, at least 80%, at least 85%, at least 90%, at least 95%, at least 97% including any range between, of the initial cell loading remains viable upon storage thereof under suitable storage conditions.

In some embodiments, suitable storage conditions comprise inter alia: ambient atmosphere and a temperature of less than 50° C., less than 40° C., less than 30° C., less than 20° C., or between 3° and 45° C., between 35 and 45° C., including any range between. In some embodiments, the suitable storage conditions comprise a temperature appropriate for maintaining viability of the cells.

In some embodiments, the viable cells are homogenously distributed (e.g., dispersed) within the particles, or are in a form of agglomerates/colonies within the particles.

In some embodiments, the particles have a predefined shape. In some embodiments, the particles are printed particles. In some embodiments, the particles are substantially spherically shaped. In some embodiments, the particles within the composition are uniformly shaped. In some embodiments, the particles are solid particles. In some embodiments, the solid particles maintain their shape (i.e. are solid) in an aqueous solution or at ambient atmosphere at a temperature up to 60° C., up to 50° C., or up to 40° C., including any range between.

A skilled artisan will appreciate that the particles may have any predefined shape. In some embodiments, the solid particles are substantially characterized by a spherical shape, an elongated shape, a rod shape, a bar shape, a needle shape, cylindrical shape, elliptical shape, cubic shape, a rectangular shape, a prism shape, a conical shape, etc. A skilled artisan will appreciate that the shape of each of the solid particles may slightly or substantially vary from a specific geometrical shape. Accordingly, the solid particles may have a spherical shape, a rod-like shape, a bar-like shape, a needle-like shape, a cylinder-like shape, a horseshoe-like shape, or ellipse-like shape, meaning that the actual shape of the particle has some deviations (e.g., at least 10%, at least 50%, or more deviation) from a perfect geometrical shape.

In some embodiments, the particles are microparticles. In some embodiments, the particles are spherically shaped microparticles. In some embodiments, each microparticle is covalently bound to a functional moiety, wherein the microparticle comprises a cross linkable biopolymer and a plurality of viable cells; the cross linkable biopolymer is at least partially crosslinked by a crosslinking agent; the functional moiety has a binding affinity to a cell; and wherein the plurality of microparticles is characterized by an average particle size between 100 μm and 1 cm, between 100 μm and 500 μm, between 100 μm and 1 mm, between 500 μm and 1 cm, between 500 μm and 1 mm, between 100 μm and 5 mm, between 500 μm and 5 mm, between 100 μm and 300 μm, between 200 μm and 500 μm, between 1 mm and 1 cm, including any range between.

In some embodiments, the particles are characterized by a density below 1 g/L (e.g. between 0.5 and 0.8 g/L). In some embodiments, the particles are characterized by a density above 1 g/L (e.g. between 1.2 and 1.8 g/L). In some embodiments, the particles are characterized by a density between 0.8 and 1.5 g/L, or between 0.5 and 1.5 g/L, including any range between.

In some embodiments, the particles are characterized by a negative surface charge (as determined by zeta potential measurements). In some embodiments, the average zeta potential value of the particles is below −13 mV (e.g. between −13 and −40 mV, between −13 and −30 mV, between −13 and −20 mV, between −13 and −15 mV, including any range between).

In some embodiments, the composition of the invention comprises microparticles (e.g. hydrogel microparticles) and optionally a suitable carrier. In some embodiments, the carrier is an aqueous composition. In some embodiments, the carrier is a cell culture medium.

In some embodiments, the microparticles of the invention substantially retain its geometrical shape (or are rigid). In some embodiments, the microparticles of the invention are solid at a temperature between −20 and 50° C., including any range between.

In some embodiments, there is provided a microparticle comprising a core comprising the scaffold of the invention; wherein microparticle further comprises the functional moiety covalently bound to the core. In some embodiments, the microparticle of the invention is obtained by crosslinking a gel comprising the cross-linkable biopolymer of the invention by the cross-linking agent, as disclosed herein.

In some embodiments, the functional moiety is bound to the microparticle via a covalent bond, wherein the covalent bond comprises an amide bond, an ester, a linker, a click reaction product, a thioether bond, a linker, or any combination thereof. In some embodiments, the functional moiety is bound to the microparticle via an ester and/or via an amide.

In some embodiments, the functional moiety is bound to at least a portion of the outer surface of the microparticle. In some embodiments, at least 0.01%, at least 0.1%, at least 1%, at least 10%, or between 1 and 50%, between 1 and 20%, between 1 and 10% of the outer surface of the microparticle is in contact with or covered by the functional moiety, including any range between.

In some embodiments, the first cell population encapsulated within the particle disclosed herein is configured to secrete a growth factor into a surrounding liquid (e.g. cell medium), so as to obtain a concentration of the growth factor within the surrounding liquid of between 100 and 1000 ng/ml, between 200 and 1000 ng ng/ml, between 200 and 500 ng/ml, between 200 and 700 ng/ml, including any range between. In some embodiments, the first cell population encapsulated within the particle disclosed herein is configured to secrete TGFβ into a surrounding liquid (e.g. cell medium), so as to obtain a concentration of TGFβ within the surrounding liquid of between 100 and 1000 ng/ml, between 200 and 1000 ng/ml, between 200 and 1000 ng/ml, between 400 and 600 ng/ml, between 200 and 700 ng/ml, including any range between.

Inventors demonstrated release of TGFβ into a cell medium at a concentration of about 500 ng/ml using CRL-1390 or CRL-2048 cells encapsulated within the particles of the invention (e.g. AGFTC beads).

### Methods

In another aspect of the invention, there is provided a method for delivering an active agent to a cell, comprising contacting the cell with the composition of the invention comprising the plurality of viable cells, under conditions suitable for binding of the cell to an outer portion of the composition; wherein the plurality of viable cells of the composition are configured to secret the active agent; and wherein the active agent is essential for activity of the cell.

In some embodiments, the plurality of cells are the first cell population disclosed hereinabove, and wherein the active agent is a growth factor (e.g. TGF-β). In some embodiments, the cell is the second cell population disclosed hereinabove. In some embodiments, the first cell population is configured to secret TGF-β, and wherein the cell is selected from any one of: Embryonic stem cells, Induced pluripotent stem cells, mesenchymal stem cells, Fibro-adipogenic progenitor cells, Muscle satellite cells, Fibroblasts, Endothelial cell, and Smooth muscle cells, including any combination thereof.

In some embodiments, the method is utilized for culturing the plurality of cells. In some embodiments, the method is utilized for culturing a tissue. In some embodiments, the method is for modifying (increasing, upregulating, or reducing) one or more cell activity (e.g. growth, proliferation, differentiation, etc.).

In another aspect of the invention, there is provided a method for printing the composition of the invention, the method comprises (1) printing the scaffold of the invention comprising the first cell population; (2) covalently binding the functional moiety to the scaffold, to obtain a modified scaffold (i.e. scaffold covalently bound to the functional moiety); and (3) contacting the second cell population with the modified scaffold, thereby attaching the second cell population to an outer surface of the modified scaffold.

In some embodiments, step (1) results in the formation of a stable (or solid) 3D structure. In some embodiments, step (1) results in the formation of a crosslinked hydrogel having a 3D shape. In some embodiments, step (1) comprises printing according to a predetermined 3D structure, or to obtain a predetermined pattern attached to a substrate disclosed above. In some embodiments, steps 1-3 are performed one or more times. In some embodiments, steps 1-3 are performed in a consecutive order (i.e. step 2 is performed after completion of step 1, etc.).

### Kit

In another aspect of the invention, there is provided a kit comprising (a) the cross linkable biopolymer as disclosed herein, (b) the functional moiety as disclosed herein, and (c) the crosslinking agent as disclosed herein; wherein (a), (a), and c) are in a form of separate compositions within the kit; optionally wherein each of separate compositions is in a form of a flowable aqueous composition suitable for printing. In some embodiments, (a), (b) and (c) are in a form of flowable aqueous compositions and are stored or dispensed in separate compartments.

In another aspect of the invention, there is provided a kit comprising (i) alginic acid, including any salt, or any conjugate thereof; (ii) one or more of the cross linkable protein(s), (iii) the crosslinking agent; wherein the kit comprises a first compartment comprising (i), (ii), or both (i) and (ii); and a second compartment comprising (iii); wherein (i), (ii) and optionally (iii) are in a form of a flowable aqueous composition suitable for printing (e.g. bio printing, or bio 3D printing). In some embodiments, the first compartment comprises the cross linkable biopolymer.

In some embodiments, the kit is a bioprinting kit. In some embodiments, the kit is for use in the printing of a cell scaffold. In some embodiments, the kit is for use in the printing of the scaffold disclosed herein. In some embodiments, the kit is for use according to the method of the invention.

In some embodiments, the kit comprises a predetermined amount of (iii). In some embodiments, the kit or combined preparation comprises a predetermined w/w ratio between (i) and (ii). In some embodiments, predetermined amount and predetermined ratio are so as to form the composition of the invention (e.g. a microparticle comprising a cross-linked hydrogel). In some embodiments, predetermined amount and predetermined ratio are so as to obtain an efficient cross-linking of the gel constituents, as disclosed herein. In some embodiments, any one of (i), (ii), and (iii) are as disclosed hereinabove.

In some embodiments, the first compartment and the second compartment are stored separately (e.g. in separate containers). In some embodiments, the kit is packaged in a multi-dispensing unit, so that the separate units can be easily combined or mixed together. In some embodiments, the kit is packaged in a container (e.g. cartridge) compatible with a printing apparatus).

In some embodiments, a w/w ratio between (i) and (ii) within the kit is between about 3:1 and about 1:3, between about 2:1 and about 1:2, between about 3:1 and about 1:1, between about 2:1 and about 1.5:1, between about 1.5:1 and about 1:1, between about 1:1 and about 1:2, between about 1:1 and about 1:1.5 including any range between. In some embodiments, a w/w ratio between (i) and (ii) within the kit is between about 3:1 and 1:1, or between about 3:1 and 2:1.

In one embodiment, the present invention provides combined preparations. In one embodiment, each of the combined preparations is in a form of a printable (or bio printable) composition. In one embodiment, the printable composition is compatible with a printing apparatus (e.g. a bio-printing apparatus, or a 3D bio-printing apparatus). In one embodiment, the printable composition is a flowable aqueous composition suitable for printing (e.g. bioprinting).

In one embodiment, “a combined preparation” defines especially a “kit” or a “kit of parts” in the sense that the combination partners (e.g. the 1^{st} and the 2^{nd} compartments) as defined above can be dosed independently or by use of different fixed combinations with distinguished amounts of the combination partners i.e., simultaneously, concurrently, separately or sequentially. In some embodiments, the parts of the kit of parts can then, e.g., be applied (e.g. into a printing apparatus) simultaneously or chronologically staggered, that is at different time points and with equal or different time intervals for any part of the kit of parts. In one embodiment, the combined preparation can be varied, e.g., in order to cope with the needs of a particular application or pest treatment, as can be readily made by a person skilled in the art. In one embodiment, the separate compartments of the kit are stored in the pack or dispenser device (e.g. in a form of cartridges).

In one embodiment, the pack or dispenser device is accompanied by instructions for application such as, dilution, dosing and/or preferred application method.

In some embodiments, the flowable aqueous composition is an aqueous solution. In some embodiments, the flowable aqueous composition is an aqueous dispersion or suspension. In some embodiments, the flowable aqueous composition is an aqueous cell suspension. In some embodiments, the flowable aqueous composition is a bioprinting ink. A skilled artisan will appreciate the exact parameters (e.g. viscosity, cell concentration, etc.) required for a flowable aqueous composition for use as a bioprinting ink in a bioprinting apparatus. For example, the exact parameters of the flowable aqueous composition which are required to promote undisturbed flow of the bioprinting ink through the tubing and printing head of the apparatus and to prevent clogging are known to a skilled artisan. Furthermore, the exact parameters of the flowable aqueous composition may vary depending on a specific printing apparatus and on the printing conditions (flow rate/printing speed, specific printing Nozzle, pressure-20 kPa, Infill density, etc).

In some embodiments, the flowable aqueous composition is characterized by a viscosity at room temperature (RT) between 10 cP and 10000 cP, between 10 cP and 100 cP, between 100 cP and 1000 cP, between 1000 cP and 10000 cP, including any range therebetween. In some embodiments, the viscosity is sufficient for forming a stable pattern on a surface, or a stable particle; wherein stable refers to the ability of the composition/particle to retain the initial shape (and/or dimensions) immediately after printing for at least 1, 10, or 20 minutes. In some embodiments, the flowable aqueous composition is characterized by cell concentration (e.g. of the first-, or of the second cell population) of below 10,000,000 cell units/ml.

In some embodiments, the kit further comprises a plurality of viable cells (i.e. the first cell population, and/or the second cell population). In some embodiments, the plurality of viable cells are in incorporated within the first compartment. In some embodiments, the viable cells are mixed together with i and ii within the first compartment. In some embodiments, the constituents of the first compartment are in a form of an aqueous composition comprising the first cell population together the cross linkable biopolymer (e.g. i and ii).

In some embodiments, the aqueous composition of the first compartment is a non-crosslinked flowable (dispensable or injectable) hydrogel comprising a mixture of the cross linkable biopolymers (e.g. alginic acid and one or more of the cross linkable protein) and the first cell population. In some embodiments, the first cell population is dispersed within the aqueous composition. In some embodiments, the aqueous composition of the first compartment is devoid of the cross-linking agent. In some embodiments, the aqueous composition of the first compartment is devoid of the functional moiety and/or of the second cell population.

In some embodiments, the first compartment comprises alginic acid, collagen and fibrinogen and, optionally further comprises at least one protein selected from gelatin, vitronectin, laminin and fibronectin. In some embodiments, the first compartment comprises alginic acid, collagen fibrinogen and gelatin, and optionally further comprises the first cell population.

In some embodiments, a w/w ratio between collagen and fibrinogen within the kit is between about 1:10 and 1:50, between about 1:10 and 1:30, between about 1:10 and 1:20, between about 1:10 and 1:40, including any range between.

In some embodiments, a w/w or w/v concentration of alginic acid within the first compartment is between about 0.5 and 10%, between about 0.5 and 5%, between about 0.5 and 3%, between about 1 and 3%, including any range between.

In some embodiments, a w/w or w/v concentration of gelatin within the first compartment is between about 1 and 10%, between about 1 and 5%, between about 2 and 10%, between about 2 and 6%, between about 2 and 5%, between about 2 and 8%, including any range between.

In some embodiments, a w/w or w/v concentration of fibrinogen within the first compartment is between about 0.1 and 5%, between about 0.5 and 5%, between about 0.5 and 2%, between about 0.5 and 3%, between about 0.5 and 1%, including any range between.

In some embodiments, a w/w or w/v concentration of collagen within the first compartment is between about 0.001 and 1%, between about 0.1 and 5%, between about 0.1 and 2%, between about 0.001 and 0.1%, between about 0.001 and 0.5%, including any range between.

In some embodiments, a w/w or w/v concentration of any of alginic acid and gelatin within the first compartment is between about 1 and about 5%; a w/w or w/v concentration of fibrinogen within the first compartment is between about 0.5 and 2%, and a concentration of the first cell population is between 500,000 and 10,000,000 cell units/ml.

In some embodiments, the functional moiety is in a form of a flowable aqueous composition within the kit. In some embodiments, the functional moiety is in a form of a flowable aqueous composition is stored within a third compartment. In some embodiments, the third compartment consists essentially of an aqueous solution of the functional moiety. In some embodiments, the functional moiety is HA and the third compartment consists essentially of an aqueous HA solution. In some embodiments, the aqueous HA solution within the third compartment comprises HA activated by an activating agent. The activating agent is configured to react with a carboxy group resulting in the formation of an active ester, which readily reacts with nucleophiles (e.g. amino-based nucleophiles) at room temperature.

The term “active ester” refers to an ester with enhanced reactivity (fast kinetics) towards a nucleophilic attack, as compared to a regular alkyl ester. Active esters react with nucleophiles at room temperature, resulting in almost quantitative amide bond formation. The active esters have alcohol component inducing greater electron withdrawing effect, as compared to a regular alkyl ester. Withdrawal of electrons enhances the electrophilic character of the carbonyl carbon and thereby facilitates the formation of the tetrahedral intermediate with the nucleophile. Usually, the OR component of an active ester is a better leaving group, as compared to OR, where R is a linear alkyl (e.g. methyl or ethyl).

Various activating agents (also known as coupon reagents such as those used for peptide synthesis) are known in the art, such as carbodiimides (e.g. EDC, DCC), phosphonium/uronium agents (e.g. PYBOP, HATU, HBTU).

In some embodiments, the aqueous HA solution within the third compartment comprises HA and an activating agent, in a weight ratio between HA and the activating agent between 1:1 and 1:10, or between 1:1 and 1:5, or between about 1:2 and 1:6, including any range between. In some embodiments, a w/w concentration of HA within the third compartment is between 0.1 and 10%, between 0.1 and 5%, between 1 and 5%, including any range between.

In some embodiments, the kit further comprises a fourth compartment comprising the second cell population. In some embodiments, the second cell population is in a form of a flowable aqueous composition within the fourth compartment. In some embodiments, a concentration of the second cell population in the flowable aqueous composition is between 500,000 and 10,000,000 cell units/ml.

In some embodiments, the second compartment comprises one or more of the cross-linking agents as disclosed above. In some embodiments, one or more of the cross-linking agents are in a form of a flowable aqueous composition within the second compartment.

In some embodiments, the second compartment comprises (i) a multivalent metal cation and (ii) thrombin, wherein the kit further comprises instructions for contacting the first compartment and the second compartment at a predetermined ratio, to obtain a crosslinked hydrogel. In some embodiments, the predetermined ratio of the multivalent metal cation is sufficient for crosslinking alginic acid, and the predetermined ratio of thrombin is sufficient for crosslinking collagen.

In some embodiments, the predetermined ratio is sufficient for obtaining a crosslinked hydrogel comprising the first cell population. In some embodiments, the crosslinked hydrogel is the scaffold of the invention. In some embodiments, the predetermined ratio comprises a weight portion of Ca2+ relative to alginate between 1 and 20%.

In some embodiments, the flowable aqueous composition of the second compartment comprises Ca²⁺ and thrombin, wherein the concentration of Ca²⁺ within the flowable aqueous composition of the second compartment is between 10 and 200 mM, between 10 and 100 mM, between 20 and 200 mM, between 20 and 100 mM, between 30 and 80 mM, or about 50 mM, including any range between. In some embodiments, the concentration of thrombin within the flowable aqueous composition of the second compartment is between 500 and 5000 thrombin units (U)/ml, between 500 and 2000 U/ml, between 700 and 2000 U/ml, between 800 and 1500 U/ml, between 800 and 2000 U/ml, including any range between.

In some embodiments, the kit further comprises instructions for binding the functional moiety (e.g. HA) to the crosslinked hydrogel, by contacting the flowable aqueous composition of the third compartment with the crosslinked hydrogel. In some embodiments, the amount of the flowable aqueous composition of the third compartment is so as to obtain a weight portion of the functional moiety (e.g. HA) ranging between 1 ng and 1 mg, between 1 ng and 10 ng, between 1 ng and 100 ng, between 1 ng and 1000 ng, or between 1 ng and 10 ug of the functional moiety per 10 mg of the crosslinked hydrogel, including any range between.

In some embodiments, the kit further comprises instructions for contacting the aqueous composition of the fourth compartment with the crosslinked hydrogel bound to the functional moiety. In some embodiments, the amount of the aqueous composition of the fourth compartment is so as to obtain a concentration of the second cell population within the scaffold of between 500 and 2000000, between 10000 and 2000000, between 50000 and 2000000, between 100000 and 2000000 cell units per 10 mg of the scaffold, including any range between.

In some embodiments, contacting is performed by printing.

In some embodiments, the kit is for manufacturing the composition of the invention (e.g. in a form of a pattern or particles) comprising the first and second cell populations stably attached thereto. In some embodiments, the kit is for manufacturing the composition of the invention comprising the first cell population secreting the sufficient amount of the active agent to allow cell growth and proliferation of the second cell population in contact with the composition, as disclosed herein. In some embodiments, the kit is for manufacturing the composition of the invention according to the method disclosed herein.

In some embodiments, the kit is for manufacturing the composition of the invention comprising the crosslinked hydrogel covalently bound to HA (in an amount ranging between 0.1 ng and 100 ng per 1 mg of the hydrogel) and comprising the first cell population configured to secret a growth factor (e.g. TGF-beta); and wherein the first cell population is embedded within the crosslinked hydrogel.

In some embodiments, the kit is for manufacturing the composition of the invention in a form of particles disclosed above; wherein an amount of the first cell population within each particle is between 1000 and 200000 cell units, and wherein an amount of the second cell population within each particle is between 500 and 2000000 cell units.

### General

As used herein the term “about” refers to +/−10%.

The terms “comprises”, “comprising”, “includes”, “including”, “having” and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure. In some embodiments, “consists essentially of” encompasses between 80 and 100%, between 80 and 99%, between 90 and 99%, between 90 and 100%, between 92 and 99%, between 93 and 99%, between 95 and 99%, between 95 and 97% between 93 and 100%, between 95 and 100%, between 97 and 99%, between 97 and 100% by weight of the composition consists of the listed constituents and is devoid of additional ingredients which contribute to the essential properties of the composition, as disclosed herein.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

As used herein, the term “substantially” refers to at least 80%, at least 85%, at least 90%, at least 92%, at least 95%, at least 96%, at least 97%, at least 98%, at least 99%, including any range or value therebetween.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Example 1

Exemplary method for the preparation of a control crosslinked hydrogel is provided hereinbelow.

The control hydrogel (AGFCH) included Sigma-Aldrich products with the combination of alginate (38.5% v/v), gelatin (38.5% v/v), fibrinogen (7.7%), collagen (7.7% v/v) and hyaluronic acid (7.7% v/v). Alginate (W201502; Sigma-Aldrich, USA) was dissolved in 10% glycerol in PBS at stock concentration of 19.6-25 mg/mL, gelatin (G9764; Bio-Basic, USA) was dissolved in 10% glycerol in PBS at stock concentration of 45-75 mg/mL. Alginate and gelatin were mixed in 1:1 ratio. Fibrinogen (F38791; Sigma-Aldrich, USA) was dissolved in PBS at stock concentration of 50 mg/mL, collagen (C9791; Sigma-Aldrich, USA) was dissolved in 0.1M acetic acid at stock concentration of 2.2 mg/mL and hyaluronic acid (08185; Sigma-Aldrich, USA) was dissolved in water at stock concentration of 2 mg/mL. Fibrinogen, collagen and hyaluronic acid were mixed in 1:1:1 ratio and added to 4×10⁶ pelleted cells after centrifugation for 10 min, in 300 g speed and after medium discarding.

### 3D Printing

The 3D model was prepared in 24 well. The gel (AGFCH) was chilled to 4 C, mixed with 4×106 HTB75 cell line and printed using Cellink bioprinter (Cellink, Gothenburg Sweden) into lens or droplets shape.

Cross-linking

Cross-linking was done using 990 μl/mLCaCl2 (A610050; Bio-Basic, USA) from stock concentration of 50 mM CaCl2 and 10 U thrombin (SRP6557; Sigma-Aldrich, USA) from stock concentration of 1000 U/mL. Cross-linking duration was for 5 min.

Specific printing conditions and applications are as described in greater detail in WO 2022153319A1.

The AGFCH was prepared and mixed with HTB75 cell line (i.e. an exemplary first cell population). Mixture was printed as micro bioreactors in the form of beads with a diameter of 200 μm.

The inventors successfully modified the printed beads with 6-aminofluorescein (modelling the surface decoration with a functional moiety in the microparticles of the invention), according to a procedure described below. Covalent binding of the AGFCH beads

Stock solutions of 30 mg of EDC and 5 mg of 6-aminofluorescein dye were prepared separately, each in 10 mL of 0.1 M MES (pH 7.6) buffer. The amine edge groups of the dye reacted with the carboxyl edge groups of the bioreactor AGFCH bead surface in the presence of EDC to form an amide bond. The solution was then mixed gently by slow mode shaker for 1 h at room temperature. Subsequently, the AGFCH bioreactor bead was taken out and washed by MES buffer several times until remove all excess reactants.

These modified beads provide an initial proof of concept for the ability to functionalize the surface of the printed micro-bioreactors (microparticles of the invention) with functional moieties. The inventors are currently in process of synthesizing microparticles covalently bound to a functional moiety capable of promoting attachment of a second cell population.

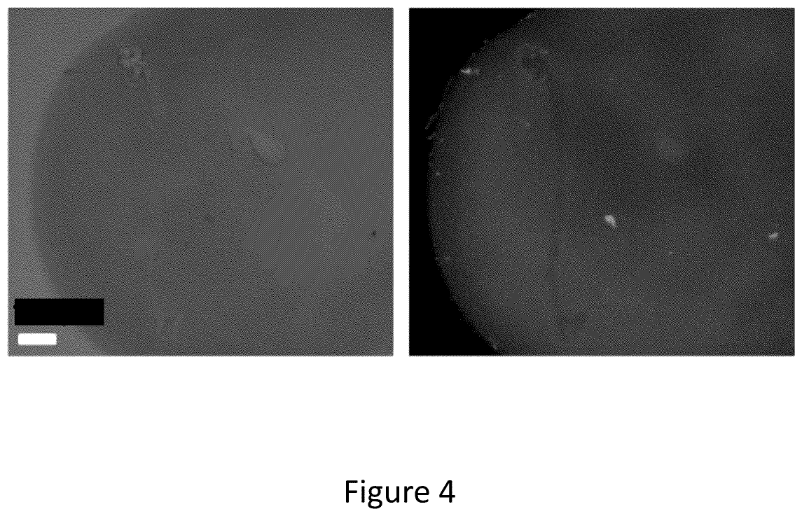

Induction of growth factors production from the printed micro-bioreactors (microparticles of the invention) has been performed as described hereinbelow. As shown in FIG. 4, the microparticles of the invention are characterized by enhanced production of growth factors (e.g. IL-8), as compared to non-induced cells.

Micro-bioreactors (HTB75 cells printed in AGFCH) were incubated for excitation with 20 ng-mL IL1β or TNFα in a range of 100-500 ng/ml with complete DMEM for 24 h, in 24-well cell culture plate. Incubation conditions were 37° C. in 5% CO2 in a humidified incubator. Following, the level of secreted IL-8 was determined. Supernatant samples were collected and tested using IL-8 ELISA kits (DY208 respectively, R&D Systems, Minneapolis, MN, USA).

Surprisingly, the inventors found that upon contacting the printed AGFCH beads with the second cell population no sufficient attachment of the second cell population was obtained.

### Example 2

HA is a Non immunogenic and biocompatible polymer, and may be suitable for the propagation of multiple cell types, including immune cells. HA binds mammalian cells via different receptors displayed on cell surface/membranes (e.g., CD44). This ability of HA to bind receptors display on cells surface is important for cell viability and proliferation. HA may also allow differential binding to cells. For example, in case of cell inflammation or stemness, CD44 is in access and with increased display on cell membranes.

Accordingly, the inventors successfully printed Alginate+gelatin+collagen+fibrinogen+thrombin (AGFTC) beads containing a first cell population (mammalian cells configured to secret TGF-beta) according to a procedure disclosed hereinbelow.

AGFTC beads were obtained via crosslinking of a hydrogel consisting of the following constituents: alginate (1.5-2.5% w/v), gelatin (3.5-5% w/v), fibrinogen (0.5-1%), collagen (0.01-0.05% v/v).

Furthermore, the AGFTC beads were covalently linked to HA (via an activating agent suitable for forming an active ester of HA, such as EDC), to obtain AGFTC-HA beads. Surprisingly, the AGFTC-HA beads facilitated attachment of the second cell population thereto. Accordingly, the AGFTC-HA can be used as a scaffold for a cell culture of the second cell population (such as but not limited to: —Embryonic stem cells, Induced pluripotent stem cells, mesenchymal stem cells, Fibro-adipogenic progenitor cells, Muscle satellite cells, Fibroblasts, Endothelial cell, and Smooth muscle cells, including any combination thereof).

### Materials and Methods

Isolation of Bovine Mesenchymal Stem Cells (BMSC) from Meat

BSC were isolated from the semi-membranous muscle tissue of a 1 year old cattle carcass according to the following protocol:

Take ˜2 g resected tissue was soaked in sterile PBS supplemented with 3% penicillin-streptomycin-nystatin solution (PSN). The tissue was washed 3× with PBS/PSN, cleaned of fat and connective tissue, and minced using a sterile tweezer/scissors. The tissue was then transferred to a sterile 50-ml centrifuge tube (Tube #1) containing 0.72 mg/ml collagenase (type 1) in 10 ml DPBS (PBS-Ca/-Mg). Filter through a 0.22 μm filter. The tube was placed in a water bath at 37° C. for 1.5 h and shaken vigorously every 10 min. Then it will be centrifuged at 200×g for 10 min and the supernatant was transferred to a new 50-ml tube (Tube #2).

Pre-warmed 0.25% trypsin-EDTA was added to Tube #1 and placed in a water bath at 37° C., for 20 min. In parallel, Tube #2 was centrifuged for 5 min at 300×g, the supernatant was discarded, and the cell pellet was resuspended in 10 ml MEM-α medium.

Once the trypsinization was completed, Tube #1 was centrifuged for 5 min, at 300×g, the supernatant was discarded and 5 ml MEM-α medium was added. Tube #1 was then centrifuged again for 5 min at 200×g. The middle phase of Tube #1 was collected and transferred to Tube #2.

Finally, the cell pool (Tube #2) was centrifuged at 500×g for 10 min, the supernatant was discarded, and the cell pellet was resuspended in a 10 ml MEM-α medium. The suspension was passed through a 40 μm strainer into 100 mm dishes and placed in the incubator at 37° C., 5% CO2, for 2 h, to remove fibroblasts. Then, the dish was gently rinsed with warmed fresh MEM-α medium and the cells were seeded in a new culture flask.

### Cell Culture Maintenance

Bovine mesenchymal stem cells (BSMC) passages were sub-cultured cell culture flasks. The sub-culture medium included BSC growth medium (described below) supplemented with 62 ngml-1 recombinant human heparin-binding epidermal growth factor-like growth factor (rhHB-EGF; R&D Systems; 259-HE), 100 ngml-1 recombinant human IGF-1 (rhIGF-1; R&D Systems; 291-G1) and 10 ngml-1 recombinant basic FGF (R&D Systems; 2099-FB).

The BSC growth medium consisted of modified Eagle's medium a (MEM-α) (Biowest), supplemented with 10% fetal bovine serum (FBS; Biowest), and 1% penicillin-streptomycin (antibiotics (Ab)/amphotericin (Am)) (Biological Industries; 03-033-1B).

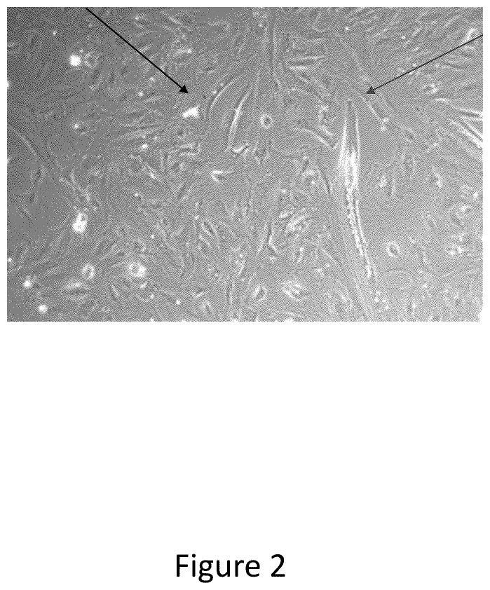

BMSC cell culture was established by the inventors. Characterization was done based on phenotypic characterization (FIG. 2)

### Preparation and Printing of Beads

Procedure for preparation of ECM Gel (Alginate+gelatin+collagen+fibrinogen+thrombin; AGFTC)

Sterilization of alginate and gelatin using UV exposure for 20 min. Wash the magnetic bead with 100% EtOH for 30 min and autoclave the magnetic beads, Scintillation Vials, 3 mL cartridges with tip caps, Nozzles, and Female/female Luer lock adapter.

Alginate solution (1.5-2.5% w/w alginate) has been prepared by dissolving a predefined amount of alginate and about 2-3 fold amount of gelatin in 1-10 ml solvent (aqueous solution with glycerol) in a sterile scintillation vial having magnetic beads. The resulting solution was heated to about 60-70° C. followed by a subsequent cooling.

Then a 1:1 molar fibrinogen and collagen aqueous mixture was prepared (concentration of fibrinogen and collagen within the solution was 50 mg/mL and 2.2 mg/mL respectively).

The crosslinking agent solution was prepared by mixing 990 ul CaCl2 (50 mM equivalent to 5.55 mg/ml of Ca²⁺) and 10 ul (10 Units, U) thrombin (to result in a concentration of thrombin of 1000 U/mL).

The first cell population (e.g. bovine epithelial cells such as CRL-1390 or CRL-2048) was harvested to generate a cell pellet having 1-10 million cells. The cell pellet was dissolved in 100-1000 μl fibrinogen and collagen mixture and transferred to a 3 ml syringe.

### Bio Printing Procedure (AGFTC Beads)

Basic setup parameters: Nozzle-25G, Speed-0.5-2 mm/sec, pressure-20 kPa, Infill density-0%, 3D model-cylinder 5X1.

### Printing of AGFTC Beads was Performed as Follows:

Take 1 ml of alginate solution in sterile 3 ml cartridges and mix thoroughly using 3 ml syringe including the mixture comprising the first cell population, fibrinogen and collagen. Rinse the wells with anti-adherent rinsing solution let them dry completely and wash them with PBS and dried. Set the print head and start printing the bead at 20 KPa. Add crosslinking agent (step 10) and incubate for 5-10 min, wash the structure with Complete media. Add 500 μl Complete media and transferred to CO2 incubator (day 1).

Digestion of AGFTC has been performed using a digestion solution containing 0.05 M sodium citrate and 0.05 M EDTA.

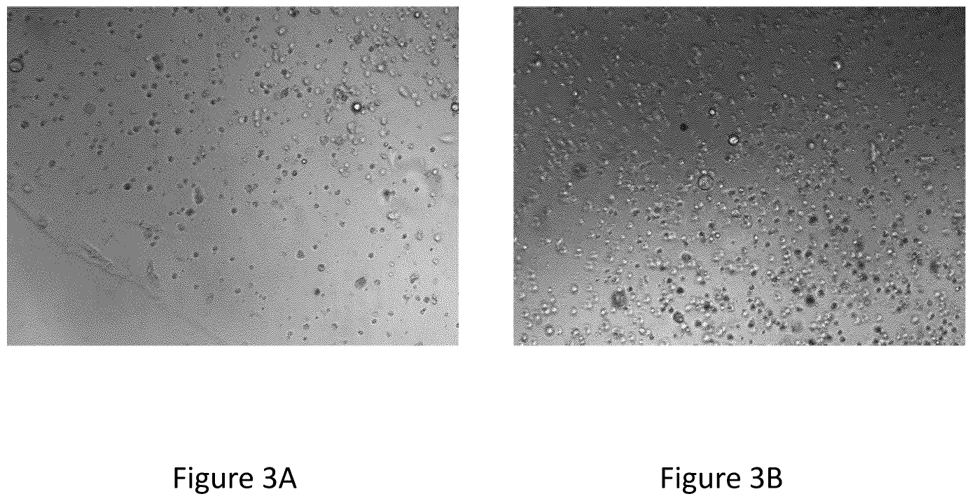

AGFTC Beads including proliferating Bovine cells CRL-1390 and CRL-2048 were successfully printed using the procedure disclosed above, as confirmed by confocal images (see FIGS. 3A-B)

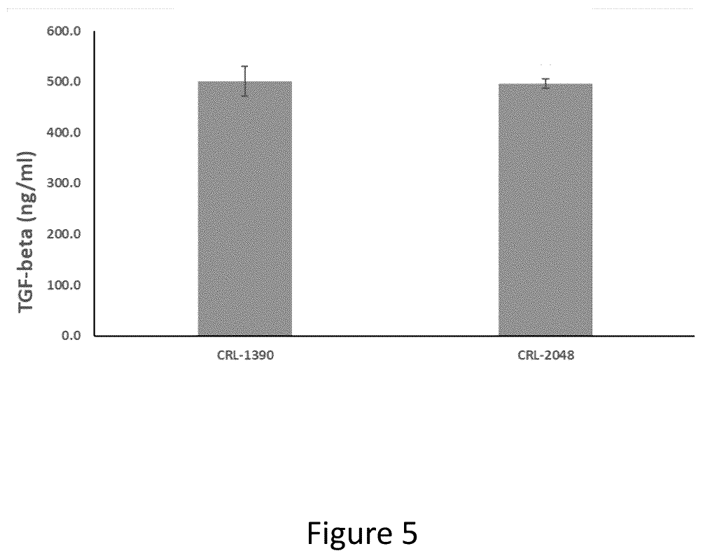

CRL-1390 and CRL-2048 encapsulated/embedded within AGFTC beads stably produced TGF1β, the main growth factor needed for cultured meat. As shown in FIG. 5 the encapsulated/embedded cells synthesized and secreted TGF1β in culture. The concertation of TGF1β in the cell culture medium was between about 300 and about 500 ng/ml (about 530 ng/ml) as determined 6-15 days after printing. Treatment with TNFα does not change TGF1β secretion from cells.

### Binding Hyaluronic Acid (HA) to the Printed AGFTC Beads

The following procedure was performed to covalently bind the functional moiety (HA) to the printed scaffold (AGFTC beads).

HA was activated (transferred into an active ester form) by reacting thereof with at least 4 fold excess by weight of activating agent (1-Ethyl-3-(3-dimethylaminopropyl) carbodiimide, EDC) in a aqueous buffer (0.1M MES, pH 4.5), followed by addition of AGFTC beads. Upon completion of the reaction the unreacted HA was washed with an aqueous buffer solution.

### Fluorescent Labeling of HA

HA was labeled by 6 amino fluorescein as follows:

Dissolve 2 mg of hyaluronic acid in 200 μl of conjugation buffer (0.1M MES, pH 4.5) and add at least 4 weight equivalents of EDC to the solution followed by addition of at least 4 weight equivalents of 6-amino fluoresceine.

The reaction was completed within 3 hours, followed by precipitation with EtOH, to obtain the labeled HA.

The inventors used the labeled hyaluronic acid to evaluate HA binding to the AGFTC beads. Successful HA binding was determined using confocal microscopy (FIG. 4).

Successful HA labeling was confirmed by drastic change of zeta potential of HA (−13 mV before labeling and +0.5 mV after labeling). A similar phenomenon was observed with the labeled hyaluronic acid attached to the AGFTC beads. Further, it is postulated that exemplary AGFTC beads of the invention covalently surface modified with pristine HA (without fluorescent labeling) will show are zeta potential shift from −5 mV before surface modification to a more negative zeta potential values.

Furthermore, the inventors confirmed a stable binding of the second cell population (e.g. Bovine mesenchymal stem cells, BMSC) to the HA-modified AGFTC beads. The inventors evaluated attachment of BMSC to HA-modified AGFTC vers. pristine (non-modified) AGFTC beads. Additionally, attachment of BMSC to HA-modified AGFTC with and without first cell population (e.g. CRL-1390) was assessed.

The tested AGFTC beads (with and without CRL-1390) were HA modified using varying amounts of HA. Then, BMSC were seeded on the tested AGFTC beads. Number of attached cells and non-attached cells was monitored at 2 and 7 days following seeding.

The results clearly demonstrate that both HA modification and inclusion of the first cell population inside the AGFTC beads are essential for superior attachment of the second cell population.

The inventors postulated that HA modification of the scaffold of the invention in an amount ranging between 1 and 100 ng/bead, or between about 10 and about 1000 ng/bead is preferential to facilitate a maximal attachment of BMSC. The abovementioned amount corresponds to between 1 and 100 ng or between 1 and 1000 ng HA per 10-50 mg of the scaffold (average mass of the bead disclosed in the Examples section is between 10-50 mg).

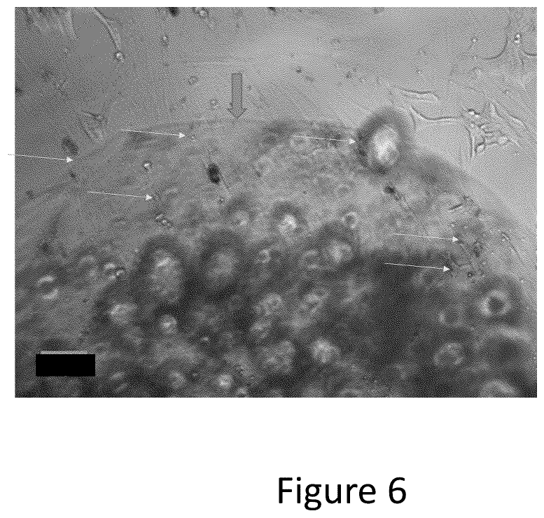

In addition, expression of marker genes associated with differentiation of the BMSC to muscle cells were examined. The results surprisingly demonstrate that BMSC attached to the scaffold of the invention (AGFTC beads) undergo cell differentiation to muscles cells without addition of growth factors and/or differentiation factors (see FIG. 6).

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A composition comprising a scaffold covalently bound to a functional moiety, wherein:
the scaffold comprises a cross linkable biopolymer and a first cell population;
the cross linkable biopolymer is at least partially crosslinked by a crosslinking agent within said scaffold;
said functional moiety has a binding affinity to a second cell population; and wherein said functional moiety comprises a hyaluronic acid including any salt and any copolymer thereof.

2. The composition of claim 1, wherein said cross linkable biopolymer comprises (i) one or more of a cross-linkable polysaccharide, (ii) one or more of a cross-linkable protein or both (i) and (ii), wherein any of (i) and (ii) includes any salt, any conjugate, or any hydrolysate thereof; wherein said second cell population is attached to the scaffold via said hyaluronic acid; and wherein said cross-linkable polysaccharide is selected from alginic acid, chitosan, gellan gum, dextran, agarose, and carrageenan; and wherein said cross-linkable protein is selected from gelatin, collagen, fibrinogen, vitronectin, laminin and fibronectin including any salt, any conjugate, or any hydrolysate thereof.

3. (canceled)

4. (canceled)

5. The composition of claim 1, wherein said covalently bound is via an amide bond, via an ester bond, a linker, a click reaction product, a thioether bond, a linker, or any combination thereof; and wherein said second cell population comprises mammalian cells selected from any one of: Embryonic stem cells, Induced pluripotent stem cells, mesenchymal stem cells, Fibro-adipogenic progenitor cells, Muscle satellite cells, Fibroblasts, Endothelial cell, and Smooth muscle cells, including any combination thereof.

6. The composition of claim 2, wherein a w/w ratio between the cross linkable polysaccharide and the cross linkable protein within said scaffold is between about 1:1 and about 1:3.

7. The composition of claim 1, wherein said crosslinking agent is selected from thrombin, a multivalent metal cation, a polyanion, and a chemical cross-linker, including any salt and any combination thereof; and wherein said scaffold is a hydrogel.

8. The composition of claim 7, wherein said cross-linkable biopolymer comprises alginic acid, gelatin, collagen and fibrinogen including any salt thereof; and wherein said crosslinking agent comprises thrombin and the multivalent metal cation; wherein said multivalent metal cation is or comprises Ca2+.

9. (canceled)

10. The composition of claim 1, wherein said scaffold is characterized by a predefined pattern on a support or by a predefined shape, and wherein said functional moiety is bound to at least a portion of the outer surface of said predefined pattern or of said predefined shape; wherein said predefined shape comprises a particle characterized by an average particle size between 100 μm and 1 cm; optionally wherein said particle is a substantially spherical particle; wherein said first cell population is embedded into or is located inside the particle; wherein said first cell population comprise mammalian cells being in a form of a cell culture, a tissue, or both; and wherein said mammalian cells are configured to secret an active agent; wherein said active agent comprises a small molecule, a growth factor, a differentiation factor, a polyamino acid, a polynucleic acid, or any combination, thereof.

11. (canceled)

12. (canceled)

13. (canceled)

14. The composition of claim 10, wherein the growth factor comprises any one of Transforming growth factor beta (TGF-β); Insulin-like growth factor 1; fibroblast growth factor 2; epidermal growth factor; interleukin 6; Neuregulin 1, or any combination thereof; and wherein said mammalian cells comprise TGF-β secreting cells; optionally wherein said TGF-β secreting cells comprise any one of a macrophage, an epithelial cell, and a monocyte, including any combination thereof; and wherein said macrophage comprises a differentiated macrophage or a non-differentiated macrophage; wherein an amount of said first cell population within said particle is between 1000 and 200000 cell units; wherein an amount of said second cell population within said particle is between 500 and 2000000 cell units; and wherein said functional moiety is hyaluronic acid, and wherein an amount of hyaluronic acid relative to said hydrogel is between 0.1 ng and 0.1 mg per 1 mg of said hydrogel.

15. (canceled)

16. (canceled)

17. (canceled)

18. A method for delivering an active agent to a cell, comprising contacting the composition of claim 1 with said cell, under conditions suitable for binding of said cell to an outer portion of said composition; and wherein said plurality of viable cells of said composition are configured to secret said active agent; and wherein said active agent is essential for activity of said cell.

19. The method of claim 18, wherein said plurality of cells comprise mammalian cells; and wherein said active agent comprises a small molecule, a growth factor, a polyamino acid, a polynucleic acid, or any combination, thereof; and wherein the growth factor comprises any one of Transforming growth factor beta (TGF-β); Insulin-like growth factor 1; fibroblast growth factor2; epidermal growth factor; interleukin 6; Neuregulin 1, or any combination thereof.

20. (canceled)

21. (canceled)

22. A kit comprising (i) alginic acid, including any salt, or any conjugate thereof; (ii) one or more of a cross linkable protein, and (iii) a crosslinking agent; wherein:
the kit comprises a first compartment comprising said (i) and (ii); a second compartment comprising said (iii);
said (i), said (ii) and said (iii) are each in a form of a flowable aqueous composition;
said kit further comprises a functional moiety having a binding affinity to a second cell population; and said kit optionally comprises a first cell population configured to secret an active agent.

23. The kit of claim 22, wherein said functional moiety comprises a carboxy group; wherein a w/w ratio between (i) and (ii) within said kit is between about 1:1 and about 1:3; wherein said (i) and said (ii) are mixed together with the first cell population within the first compartment; wherein said kit further comprises instructions for contacting said first compartment and said second compartment at a predetermined ratio, to obtain a crosslinked hydrogel; and wherein said first cell population is embedded within said crosslinked hydrogel.

24. (canceled)

25. (canceled)

26. The kit of claim 23, wherein said functional moiety comprises hyaluronic acid; and wherein said functional moiety is stored within a third compartment further comprising a sufficient amount of an activating agent and wherein said contacting is performed by printing; and wherein said activating agent is configured to promote a covalent bonding between said functional moiety and said crosslinked hydrogel; and wherein said kit further comprises instructions for contacting said third compartment with said crosslinked hydrogel, to obtain said functional moiety covalently bound to said crosslinked hydrogel; wherein an amount of the functional moiety is between 1 ng and 1 mg per 10 mg of said crosslinked hydrogel.

27. (canceled)

28. (canceled)

29. The kit of claim 22, wherein said crosslinking agent is selected from thrombin, fibrinogen, a multivalent metal cation, a polyanion, and a chemical cross-linker, including any salt and any combination thereof; optionally wherein said multivalent metal cation comprises Ca²⁺; wherein the one or more of a cross linkable protein comprises collagen, gelatin and fibrinogen, and optionally further comprises at least one protein selected from vitronectin, laminin and fibronectin, including any salt, any conjugate, or any combination thereof; and wherein said crosslinking agent comprises Ca²⁺ and thrombin; wherein a w/w ratio between collagen and fibrinogen within said kit is between about 1:10 and 1:50.

30. (canceled)

31. (canceled)

32. (canceled)

33. The kit of claim 29, wherein the first compartment comprises alginic acid and gelatin, each independently at a concentration between about 1 and 10% w/w, and further comprises collagen and fibrinogen; wherein a concentration of fibrinogen is between about 0.1 and about 5% w/w; and further comprises the first cell population; and wherein the second compartment comprises Ca²⁺ at a concentration between 10 and 200 mM and thrombin at a concentration between 500 and 5000 U/ml.

34. (canceled)

35. A crosslinked hydrogel obtained by contacting the first compartment and the second compartment of the kit of claim 22, wherein said crosslinked hydrogel comprises a first cell population configured to secret an active agent; and wherein said first cell population is embedded within said crosslinked hydrogel.

36. The crosslinked hydrogel of claim 35, being in a form of a particle or in a form of a pattern on top of a substrate; wherein said particle is characterized by a predefined shape and further comprising a functional moiety covalently bound to said particle; and wherein the functional moiety has a binding affinity to a second cell population.

37. (canceled)

38. The crosslinked hydrogel of claim 36, wherein the functional moiety comprises hyaluronic acid, including any salt thereof.

39. (canceled)

40. (canceled)

41. (canceled)

42. (canceled)

43. (canceled)

44. (canceled)
